(19) United States
(12) Patent Application Publication
(10) Pub. No.: US 2024/0180625 A1
(43) Pub. Date: Jun. 6, 2024

(54) IMAGE GUIDED DELIVERY OF COMPOSITIONS AND RELATED METHODS

(71) Applicant: RevBio, Inc., Lowell, MA (US)

(72) Inventors: Brian J. Hess, Charlestown, MA (US); George W. Kay, Sharon, MA (US); Michael C. Brown, Harvard, MA (US); Brittany McDonough, Nashua, NH (US); Sourabh Boruah, Lowell, MA (US); Kevin T. Foley, Germantown, TN (US)

(21) Appl. No.: 18/283,642

(22) PCT Filed: Mar. 23, 2022
(86) PCT No.: PCT/US2022/021568

§ 371 Date: Sep. 22, 2023

Related U.S. Application Data
(60) Provisional application No. 63/164,703, filed on Mar. 23, 2021.

(51) Int. Cl. A61B 34/20; A61B 17/00; A61B 90/00; A61L 24/00; A61L 24/02

(52) U.S. Cl. CPC A61B 34/20; A61B 17/00491; A61B 90/06; A61B 90/361; A61B 90/37; A61L 24/0015; A61L 24/02; A61B 2017/00119; A61B 2017/00495; A61B 2034/2051; A61B 2090/064; A61B 2090/367; A61B 2090/374; A61B 2090/3762; A61B 2090/378; A61L 2400/06; A61L 2430/02

(57) ABSTRACT

The present disclosure features, inter alia, compositions and systems for delivery of adhesive compositions to a site in a subject utilizing image guided delivery, as well as related methods.

### CLAIM OF PRIORITY

This application claims priority to U.S. Provisional Application No. 63/164703, filed on Mar. 23, 2021. The entire contents the foregoing application is hereby incorporated by reference.

### BACKGROUND

Bone injuries, including fractures, are an important public health concern, particularly for the elderly population. Evidence indicates that the incidence of bone injuries increases with age, due to factors including decreased bone health and increased risk of trauma (e.g., falls, etc.). Falls from standing height and other low-energy trauma account for nearly of all orthopedic fractures. There are multiple courses of treatment for fractures currently in use, including external fixation, percutaneous pinning with Kirschner wires, and conventional open reduction internal fixation (ORIF) procedures that involve the use of locking volar plates. These methods and procedures carry risks, with significant complications which may be amplified by underlying conditions, such as osteoporosis. Complications include graft dislodgement and subsidence of the restored bone, graft migration or cement extravasation during injection, loss of implant fixation, and junctional kyphosis. Adhesive compositions provide an attractive alternative to treatment methods solely relying on hardware; however, delivery methods of these adhesive compositions can be cumbersome and invasive. Thus, there is a need for improved methods for delivery of these compositions to wound sites, which may improve therapeutic outcomes.

### SUMMARY

The present disclosure features, inter alia, compositions and systems for delivery of adhesive compositions to a site in a subject utilizing image guided delivery, as well as related methods. In particular, the present disclosure provides systems and methods of image guided delivery of a composition to a site using percutaneous delivery, e.g., using a syringe delivery system, a trocar, etc. In some embodiments, the site is a wound site. In some embodiments, the wound site is a bone, or a plurality of bones. In some embodiments, the wound site is a bone fracture, or a gap between two or more bone fragments. Image guided delivery is envisioned to have a number of benefits over traditional delivery methods, which comprise direct visualization of the delivery site, e.g., by the naked eye. For example, image guided delivery may allow more precise control over the amount of the composition delivered to a site (e.g., a wound site). In addition, image guided delivery may allow for a minimally invasive procedure or percutaneous delivery, compared with traditional open delivery methods, therefore leading to fewer post-operative complications, faster healing times, or a reduced chance of infection. Minimally invasive procedures and percutaneous delivery of compositions (e.g., adhesive compositions) are desirable due to the reduced risk of disturbing surrounding tissues, such as vasculature or nerves, as bone regeneration processes, i.e., osteostimulative, osteopromotive or osteoinductive processes, occur following delivery of the adhesive composition. In some embodiments, image guided delivery enables real time visualization of delivery of a composition at the wound site.

The present disclosure provides a composition to be delivered to a wound site. In some embodiments, the composition is an adhesive composition. The adhesive composition may further have therapeutic properties, e.g., bone regenerative and/or bioresorbable properties. In some embodiments, the adhesive composition comprises pain control properties, anti-inflammatory properties, and/or antimicrobial properties.

The present disclosure further provides delivery systems and methods for storage, mixing, and delivery of an adhesive composition, e.g., a therapeutic composition, a bone regenerative composition, and/or bioresorbable composition, to a wound site in a subject, e.g., a delivery system comprising a cannula for percutaneous delivery.

According to an aspect of the present disclosure, a method of image-guided delivery of an adhesive composition, e.g., a therapeutic composition, bone regenerative composition, and/or bioresorbable composition, to a wound site (e.g., a bone site, e.g., a bone fracture site) includes acquiring image data (i.e., anatomical data convertible to an image observable by an operator) of the wound site, positioning a device (e.g., a syringe or trocar) to deliver the adhesive composition, e.g., the therapeutic composition, bone regenerative composition, and/or bioresorbable composition to the wound site, and delivering the composition to the wound site via the device (e.g., the syringe or trocar or specialized cannula) with a small incision in the soft tissue, e.g., percutaneous delivery. In some embodiments said acquired image data is rendered as an image in a 2D space such that delivery of the adhesive composition, e.g., therapeutic composition, bone regenerative composition, and/or bioresorbable composition, can be tracked via the real-time image displayed in the 2D space. In some embodiments, multiple acquired images can be arranged, e.g., stacked, to create a 3D image for monitoring delivery of the adhesive composition, e.g., therapeutic composition, bone regenerative composition, and/or bioresorbable composition, to the wound site. Said image data may be acquired via an imaging device, e.g., a modified fluoroscopic with multi-planar fluoroscopy, a fluoroscopic imaging device comprising an O-arm, or an ultrasound imaging device. The image(s) to be captured may be depicted in the registration field via a contrast agent, e.g., a barium containing agent (e.g., BaSO₄), present in the adhesive composition, e.g., therapeutic composition, bone regenerative composition, and/or bioresorbable composition, or via a registration element located on or along a component, e.g., a shaft or tip, of the device, e.g., a cannula. In some embodiments, an outline of the delivery device, including the tip of the delivery device, e.g., cannula, is projected on the imaging system monitor for a user or operator to visualize the delivery device with respect to the patient's tissue, e.g., wound site, based on a computer aided design file that is registered for the delivery device and related based on the registration element located on the delivery device.

According to another aspect of the present disclosure, a method of joining two or more bone fragments together at a bone site may include acquiring image data (i.e., anatomical data convertible to an image observable by the user or operator) of the bone site, positioning a device (e.g., a syringe, trocar, or cannula) to deliver an adhesive composition, e.g., therapeutic, bone regenerative and/or bioresorbable composition, to the bone site, and delivering the composition to the bone site via the delivery device (e.g., the syringe, trocar, or cannula) while receiving feedback information regarding the volume, appropriateness of the location and rate of delivery in clinically relevant time frame. For example, a clinically relevant time frame may be a continuous feedback loop, or updated on the order of seconds, e.g., every 2 seconds, or every 3 seconds, or every 5 seconds.

The adhesive composition, e.g., therapeutic composition, bone regenerative composition, and/or bioresorbable composition, may be used alone to support and fixate the fracture or it may be used in combination with hardware fixation or support devices. Exemplary hardware may include k-wires, plates, screws, anchors, rods, nails, cages, or mesh and said hardware may be metallic (e.g., stainless steel, titanium or nitinol) or a polymer (e.g., polyether ether ketone (PEEK)). In some embodiments, the delivery system enables percutaneous delivery of the prepared adhesive composition. In some embodiments, the delivery system enables percutaneous delivery of the hardware devices. In some embodiments, the delivery system is used to stabilize or anchor the hardware devices to bone.

In some embodiments, the adhesive composition, e.g., therapeutic composition, bone regenerative composition, and/or bioresorbable composition, is self-setting and resorbable over time so as to be replaced by native bone. In some embodiments, the adhesive composition has a therapeutic effect to cause bone regeneration, e.g., is osteostimulative, osteopromotive or osteoinductive. In some embodiments, the adhesive composition, e.g., therapeutic composition, bone regenerative composition, and/or bioresorbable composition, comprises a porosity and stiffness matching that of the native bone upon curing, thereby preventing stress shielding and subsidence. In some embodiments, the adhesive composition comprises a multivalent metal salt, an organic compound (e.g., a compound of Formula (I), Formula (II), Formula (III), Formula (IV), Formula (V), or Formula (VI)), and an aqueous medium. In some embodiments, the compound chosen, e.g., Formula (IV), is phosphoserine. In some embodiments the multivalent metal salt comprises calcium phosphate, calcium nitrate, calcium citrate, calcium carbonate, calcium silicate, magnesium phosphate, sodium silicate, lithium phosphate, titanium phosphate, strontium phosphate, barium phosphate, zinc phosphate, calcium oxide, magnesium oxide, or a combination thereof. In some embodiments, the adhesive composition further comprises a second agent, such as a contrast agent, e.g., BaSO₄. In some embodiments, the adhesive composition may further comprise an additive, such as a polymer, e.g., poly lactic-glycolic acid (PLGA). In some embodiments, the additive may comprise a solidified form of the adhesive composition, e.g., therapeutic composition, bone regenerative composition, and/or bioresorbable composition. In some embodiments, the additive is in the form of a microfiber. In some embodiments, the microfibers have an aspect ratio from 2:1 to 100:1. In some embodiments, the microfibers have a length of less than about 5.0 mm. In some embodiments, the components of the adhesive composition are provided as a dry powder, wherein the dry powder may be provided in a pelletized form. In some embodiments, the dry components of the composition are provided as preformed granules. In some embodiments, the preformed granules have a mean size of about 0.05 mm to about 5.0 mm. In some embodiments, said granules are porous and comprise channels on the granule surface. In certain embodiments, the channels on the granule surface are in fluid communication. In certain embodiments, the channels on the granule surface are not in fluid communication.

In another aspect, the present disclosure also features a method of delivering a therapeutic composition, e.g., adhesive composition, bone regenerative composition, and/or bioresorbable composition where the therapeutic composition is provided as part of a kit. In some embodiments, the therapeutic composition when prepared is capable of being sterilely delivered in a non-sterile field.

In another aspect, the present disclosure features a method of use comprising the steps of: anatomically reducing and aligning of one or more bones at a wound site, e.g., a fracture site; mixing the components of a adhesive composition, e.g., therapeutic composition, bone regenerative composition, and/or bioresorbable composition, e.g., by using a mixing system as described herein; utilizing the delivery system to transfer the adhesive composition from one vessel to another; and delivering the adhesive composition to the wound site (e.g., percutaneously). In some embodiments, the method further comprises monitoring the delivery of the adhesive composition via imaging of a second agent present in the adhesive composition (e.g., via fluoroscopic imaging or ultrasound imaging). In some embodiments, the method further comprises one or more additional steps including preparing (e.g., cleaning) the wound site to remove unwanted material, e.g., bone marrow or lipids from the surface of the bone (e.g., via debriding or irrigating the site) and/or applying a cast or brace to support stand-alone fixation of the therapeutic composition.

In some embodiments, for delivery of the adhesive composition, e.g., therapeutic, bone regenerative and/or bioresorbable composition to a wound site, e.g., fracture site, a cannula tip is attached to a port of a first vessel. In some embodiments, a percutaneous cut may be made at the wound site prior to attaching the cannula tip to the delivery system. In some embodiments, a percutaneous cut is made at the wound site following attachment of the cannula tip, and the cannula tip placed into the cut to deliver the adhesive composition out of the first vessel through the cannula tip. In some embodiments, the adhesive composition is delivered by applying a force on a plunger of the delivery system. In some embodiments, the adhesive composition is flowable through the cannula tip for percutaneous delivery to the fracture fixation site on the order of minutes, e.g., less than 2 minutes, 2 to 5 minutes or 5 to 8 minutes. In some embodiments, the delivery of the adhesive composition is monitored via imaging (e.g., fluoroscopic imaging) of a component (e.g., a contrast agent, e.g., BaSO₄) in the adhesive composition. In some embodiments, upon delivery of the adhesive composition, the wound site, e.g., bone fracture site, is supported by addition of a brace or cast to aid in fixation of the adhesive composition.

In another aspect, the present disclosure further provides a cannula comprising a cannula tip selected specifically for delivery of a adhesive composition, e.g., therapeutic composition, bone regenerative composition, and/or bioresorbable composition. In some embodiments, a position of the cannula is adjustable, e.g., the cannula can be guided to a specific position. In some embodiments, adjustability of the cannula is related to a telescoping function of the cannula tip to allow for the flow of the adhesive composition through the cannula to be directed to a specific location and re-directed. In some embodiments, adjustability of the cannula is related to elastic deformability of a wall of the cannula, e.g., increasing or decreasing a length of the cannula wall. In some embodiments, the cannula is constructed and arranged for one or more axes of motion, e.g., uni-/bi-/or multi-modal motion. In this configuration, the one or more axes of motion permit a broad range of motion of the cannula. For example, the motion may be deflection of the cannula tip. In some embodiments, the motion may include direction control of the full cannula. In some embodiments, said cannula comprises a nonstick surface to reduce friction between the cannula and delivery site during injection of the adhesive composition. In some embodiments, the nonstick surface comprises polytetrafluorethylene (PTFE). In some embodiments, the cannula is comprised of a hydrophobic coating along the interior. In some embodiments, the cannula comprises a nonstick or hydrophobic interior lining.

In some embodiments, a delivery system, e.g., as described herein, comprises a registration element, e.g., on the shaft or body of the delivery system or on the cannula tip such that the cannula tip is identifiable by the imaging modality as described herein. In some embodiments, said registration element comprises a microchip which is registered by the imaging modality as described herein, e.g., fluoroscopic imaging, ultrasound imaging or some other such method. In some embodiments, the outline of the delivery device, including the tip of the delivery device, e.g., cannula, is projected on the imaging system monitor for the user to visualize it with respect to the patient's tissue, e.g., wound site, based on a computer aided design file that is registered for the delivery device and related based on the registration element located on the delivery device. In some embodiments, said registration element comprises a contrast agent, e.g., BaSO₄, which is visible via the imaging modality as described herein, e.g., fluoroscopic or ultrasound imaging. In some embodiments, the tip of the cannula includes a radiopaque portion and is visible under real-time fluoroscopic or other imaging methods. More specifically, said cannula tip may be molded from a medical grade biocompatible polymer, e.g., polyethylene, blended with a radiopaque compound. Exemplary radiopaque compounds include compounds comprising barium or bismuth, e.g., barium sulfate (BaSO₄), bismuth subcarbonate ((BiO)₂CO₃), bismuth oxychloride (BiOCl), or bismuth trioxide (Bi₂O₃). In some embodiments, the contrast coloring of the cannula is lighter than the contrast coloring of the adhesive composition, e.g., therapeutic composition, bone regenerative composition, and/or bioresorbable composition, being delivered through the cannula, visually distinguishing the cannula and adhesive composition. In some embodiments, the cannula comprises a nonstick surface, e.g., low friction polymers or elastomeric polymers, and a radiopaque compound for real time visualization in the imaging field. A number of other registration mechanisms suitable to fit on the cannula tip may be used at or near the tip of the cannula, e.g., the orifice where from the adhesive composition will be delivered. In some embodiments, delivery of a adhesive composition to the fracture site, e.g., as described herein, can be monitored in real time using the registration element disposed at or near the tip of the cannula. In some embodiments, monitoring in real time the delivery of an adhesive composition permits adjustment of the direction of flow and motion of the cannula tip. In some embodiments, monitoring in substantially real time permits directional control of delivery of the adhesive composition when used in conjunction with the cannula.

The present disclosure further provides that a non-invasive device or guide may be provided to facilitate correct positioning and fixation of the fractured bone in a set position, e.g., in a holding device, in preparation for delivery of n adhesive composition. In some embodiments, said holding device maintains the position of the fractured bone site to permit delivery of an adhesive composition, e.g., as described herein, via a cannula without needing to hold the fracture site steady or necessitating the need of another party to assist in holding the position of the fractured bone site. In some embodiments, the holding device is registered via the imaging modality, e.g., fluoroscopic or ultrasound imaging to establish a reference coordinate position with respect to the wound site. For example, holding device may comprise a computer chip or metallic component that is registered via the imaging modality, e.g., fluoroscopic or ultrasound imaging. In some embodiments, the holding device is used to control and adjust the position of the wound site for greater user control of delivery of the adhesive composition to the bone fracture site. In some embodiments, an exemplary holding device is able to guide a delivery device, e.g., a cannula, wherein the holding device comprises a tube through which the specialized cannula is guided to the wound site for greater user control. The present disclosure provides that the adhesive composition, once cured, is of sufficient strength to support the wound site without need for any additional fixation aids. In some embodiments, upon complete delivery of the adhesive composition, the site is supported by addition of a brace or cast to aid in fixation of the adhesive composition.

### BRIEF DESCRIPTION OF THE DRAWINGS

FIG. 1 is a flowchart according to an exemplary method of use provided within present disclosure;

FIG. 2 shows a fluoroscopic image guided delivery view of a kyphoplasty site;

FIG. 3 shows a surgeon performing an animal cadaver kyphoplasty with image guidance via fluoroscopy;

FIG. 4 shows a close-up view of image guided kyphoplasty;

FIGS. 5A-5C show material resorption and bone replacement in critical-size rabbit model over a 52-week period, with FIG. 5A depicting 8 weeks, FIG. 5B depicting 26 weeks, and FIG. 5C depicting 52 weeks.

FIGS. 6A-B show fluoroscopic images before (left) and after (right) the percutaneous delivery of an exemplary adhesive composition to distal radius fracture.

FIG. 7 shows average compressive failure load of an exemplary adhesive composition at a distal radius fracture site compared to plate and screw fixation and Norian.

FIG. 8 shows an image of compressive failure load testing of a distal radius fracture site in a human cadaveric wrist.

FIG. 9A-D depict multiple views of an exemplary cannula tip, having four holes for controlling the delivery of an exemplary adhesive composition as described herein.

FIG. 10A-C depict multiple views of another exemplary cannula tip having eight holes for controlling the delivery of an exemplary adhesive composition as described herein.

FIG. 11A-B depict a lateral view and a head-on view of an exemplary cannula tip, having an elongated tip with holes spaced laterally across the tip, for controlling the delivery of an exemplary adhesive composition as described herein.

FIG. 12A-B depict a lateral view and a head-on view of an exemplary cannula tip, having no holes along the tip, for controlling the delivery of an exemplary adhesive composition as described herein.

### DETAILED DESCRIPTION

The present disclosure features a method of image-guided delivery of an adhesive composition, e.g., therapeutic, bone regenerative and/or bioresorbable composition, to a site (e.g., a bone fracture site). The method comprises (i) acquiring image data (e.g., via fluoroscopic, tomographic, or ultrasound imaging) of the site; (ii) positioning a device (e.g., a syringe, trocar, or cannula) to deliver the adhesive composition to the site, or (iii) delivering the adhesive composition to the site via the delivery device (e.g., the syringe, trocar or, cannula). In some embodiments, the method comprises (i) and (ii). In some embodiments, the method comprises (i) and (iii). In some embodiments, the method comprises (i), (ii), and (iii).

In another aspect, the present disclosure features a method of joining two or more bone fragments together at a wound site, wherein said method includes: acquiring image data (e.g., via fluoroscopic, tomographic, or ultrasound imaging) of the bone site, positioning a device (e.g., a syringe or trocar or cannula) to deliver an adhesive composition, e.g., therapeutic composition, bone regenerative composition, and/or bioresorbable composition, to the wound site, and delivering the adhesive composition to the wound site via the device (e.g., the syringe, trocar, or cannula).

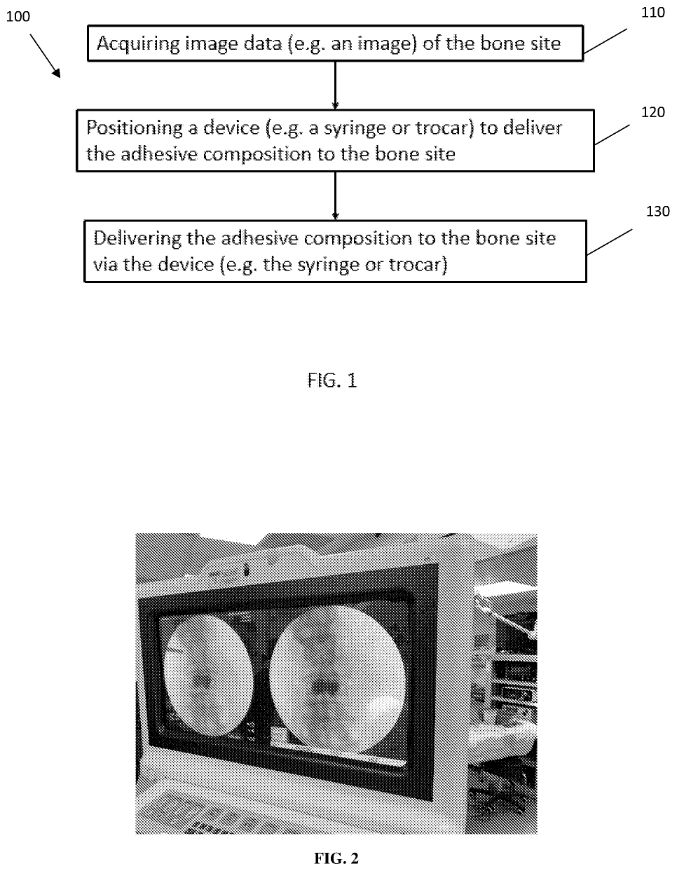

FIG. 1 shows a block diagram 100 that may be used for methods of image-guided delivery of an adhesive composition, e.g., therapeutic composition, bone regenerative composition, and/or bioresorbable composition, described herein to a wound site (e.g., a bone fracture site), and/or for methods of joining two or more bone fragments together at a wound site. In some embodiments, the methods include acquiring image data (e.g., via fluoroscopic, tomographic or ultrasound imaging) of the bone site at block 110, positioning a device (e.g., a syringe, trocar, or cannula) to deliver the adhesive composition to the wound site at block 120, and delivering the adhesive composition to the wound site via the delivery device (e.g., the syringe, trocar, or cannula) at block 130.

The present disclosure further provides a delivery system for use in delivering an adhesive composition, e.g., a therapeutic, bone regenerative and/or bioresorbable composition, to a wound site, e.g., a bone fracture site. In some embodiments, said delivery system comprises a delivery device, an adhesive composition to be delivered, and a system for acquiring image data for image guided delivery of the adhesive composition. The system may further include a non-invasive device or guide to facilitate correct positioning and holding of the fractured bone in a set position in preparation for delivery of an adhesive composition, e.g., a holding device.

### Delivery System

The present disclosure features a delivery system for preparing and delivering an adhesive composition, e.g., therapeutic composition, bone regenerative composition, and/or bioresorbable composition, to a wound site, such as a bone fracture site. In some embodiments, the delivery system is used for fixating a fracture wherein the delivery system utilizes a first vessel and second vessel for mixing and delivering an adhesive composition, e.g., percutaneous delivery. Delivery of said adhesive composition may be monitored and/or controlled via monitoring of a contrast agent present in the adhesive composition. The controlled delivery of an adhesive composition using image guidance by monitoring a contrast agent provides for several benefits. The controlled delivery of the composition may reduce the likelihood of composition extravasation or leakage from the site (e.g., wound site) where the composition is delivered. It is known that leakage of certain compositions, such as bone cement compositions, can result in damage to surrounding tissue, such as an embolisms. The present delivery system and adhesive composition may mitigate these risks by permitting the application of a controlled amount of the adhesive composition to a precise location at a site, e.g., within a subject. The adhesive composition, e.g., therapeutic composition, may be injectable through a device, e.g., a cannula at certain viscosity. For example, an adhesive composition in the fluid state may have a viscosity of at least 1,000 cP, and thus have sufficient cohesiveness to resist washout or migration due to a wet or blood-filled implantation or wound site. This cohesion may also prevent the powder and liquid components of the adhesive composition from separating, each of which may extravasate into surrounding tissue. These separated components of the composition may enter the circulatory system of the subject and cause further damage. For example, circulating components of the adhesive composition if not substantially cohesive may travel to the lungs and increase the risk of pulmonary embolism if such separation is induced by injection forces (e.g., forces exceeding 100 N) to deliver the adhesive composition to the site

In some embodiments, the delivery system is used to fixate fractures in a long bone, e.g., a bone having a tubular shaft and articular surface at each end (e.g., humerus, radius, ulna, femur, tibia or fibula.). In some embodiments, the delivery system is used to fixate fractures of a short bone, e.g., metacarpals or metatarsals. In some embodiments, the delivery system is used to fixate fractures of a flat bone, e.g., scapula, rib, or sternum. In some embodiments, the delivery system is used to fixate fractures of an irregular bone, e.g., the vertebral column or patella. In an embodiment, the delivery system is used to fixate any fracture of the types according to AO classification.

According to one aspect of the present invention, there is disclosed a delivery device for preparing and delivering an adhesive composition, e.g., therapeutic composition, bone regenerative composition, and/or bioresorbable composition, to a wound site, e.g., a bone site, e.g., a bone fracture site. In some embodiments, the delivery device includes a first vessel having a distal end and a proximal end for holding and advancing the therapeutic composition. In some embodiments, the first vessel includes a first port at the distal end through which the adhesive composition is dispensed from the first vessel. In some embodiments, the first port is connected to a lumen at the distal end that extends to a wound site, e.g., bone site, where the adhesive composition is to be delivered. In some embodiments, the first vessel includes a second port at the proximal end through which the first chamber is filled with the adhesive composition, e.g., therapeutic composition, bone regenerative composition, and/or bioresorbable composition.

In some embodiments, the delivery system includes a second vessel. In some embodiments, the first vessel of the delivery device comprises one component of the adhesive composition and the second vessel comprises another component of the adhesive composition.

In some embodiments, the first vessel is a syringe or trocar. In some embodiments, the first vessel is a cannula. In some embodiments, the cannula is rigid and able to move tissue or debris, e.g., bone fragments, around the wound site, e.g., bone fracture site. In some embodiments, the first vessel has a body and a first plunger that is movable within an inner chamber of the body. In some embodiments, the body has a second end that includes a port (e.g., an exit port). In some embodiments, movement of the first plunger relative to the body of the first vessel along a direction between the distal end and the proximal end permits contents to be advanced through the first port. In some embodiments, the first vessel and second vessel are detachable from each other once the therapeutic composition has been transferred to delivery vessel. In some embodiments, the exit port is configured to be attached to a cannula tip upon detachment from a connector and the second vessel. The composition may be delivered to a site through the cannula tip, e.g., by a minimally invasive procedure, e.g., percutaneous delivery, with the aid of image-guidance, e.g., fluoroscopy, mechanical, or computerized tomography.

In some embodiments, the second port, e.g., the exit port, is positioned to not obscure view of the delivery of the adhesive composition. In some embodiments, the second port is located on a side of the body of the second vessel. In some embodiments, the second port includes a check valve to allow fluid to flow into the inner chamber through the second port but prevent fluid from flowing out of the inner chamber through the second port. A cap may be secured to close the second vessel. In some embodiments, the cap has threads that engage threads on the body of the second vessel to secure closure.

In some embodiments, the second plunger is a dual function plunger. In some embodiments, the dual function plunger includes a plunger body and at least one mixing blade. In some embodiments, each mixing blade is movable between a first configuration, e.g., a retracted configuration, and a second configuration, e.g., a deployed configuration. When in the deployed configuration, the at least one mixing blade may be used to mix wet and dry components of a therapeutic composition, e.g., adhesive composition, bone regenerative composition and/or bioresorbable composition, within the inner chamber of the second vessel. In some embodiments, the at least one mixing blade is configured to be moved to the deployed configuration relative to the plunger body when the dual function plunger body is linked to an actuator on a mixing base.

In certain embodiments, the adhesive composition to be delivered by the delivery system does not require mixing. In this configuration, the adhesive composition may be a pre-mixed or ready to use formulation stored in a pre-filled first vessel. The conditions of storage of a pre-mixed or ready to use adhesive composition are such that the adhesive composition does not dry and/or become exposed to air prior to delivery to the wound site, e.g., sealed in the first vessel.

In some embodiments, the first chamber further comprises an advancing component for advancing the adhesive composition, e.g., adhesive, bone regenerative dual function, and/or bioresorbable composition, from the first chamber through the first port. In some embodiments, the advancing component is a manually movable element. In some embodiments, the first chamber is defined in a body of a syringe wherein the advancing component is a plunger of the syringe. In some embodiments, the advancing component is a pump.

In some embodiments, a trocar needle assembly is used as a delivery device to deliver the composition to the wound site. In some embodiments, the trocar needle assembly comprises a small, pointed awl, a cannula, and a seal. In some embodiments, use of a trocar needle assembly permits subsequent placement of another surgical instrument, e.g., a cannula, to deliver an adhesive composition, e.g., therapeutic composition, bone regenerative composition, and/or bioresorbable composition. Use of a trocar needle assembly may permit the release of residual gases from the delivery system and wound site.

The present disclosure provides a cannula that may be used to deliver an adhesive composition, e.g., therapeutic composition, bone regenerative composition, and/or bioresorbable composition, which is adjusted and/or guided. In some embodiments, the adjustability and guidance of the cannula permits control over delivery of the adhesive composition to the wound site, as described herein. In some embodiments, the cannula is made from metal. In some embodiments, an inner contacting surface, i.e., a lumen, of the cannula is made from a non-stick surface (e.g., TEFLON®) or includes a surface treatment that minimizes the friction or drag force during the administration of an adhesive composition. In some embodiments, the non-stick surface is a low friction polymer or elastomeric polymer, e.g., PTFE. Such a surface may reduce friction between the interior walls of the cannula and the composition to be delivered, e.g., a adhesive composition, e.g., therapeutic composition, bone regenerative composition, and/or bioresorbable composition, as described herein. In some embodiments, the interior surfaces of the cannula comprises a hydrophobic coating. In some embodiments, the cannula comprises a non-stick or hydrophobic removable lining. In some embodiments, a low-friction or hydrophobic interior of the cannula provides lower resistance when delivering the adhesive composition such that the therapeutic composition does not stick to the interior walls of the cannula. In some embodiments, the adjustability of the cannula is related to a telescoping ability of the cannula tip permitting the flow of an adhesive composition to be directed or re-directed to a bone fracture site. In some embodiments, adjustability of the cannula is related to the elastic deformability of the cannula wall, resulting in the shortening or extension of the length of a side wall of the cannula. In some embodiments, the cannula tip comprises a singular port or orifice through which an adhesive composition is delivered. In some embodiments, the cannula tip may comprise one or more ports or orifices through which an adhesive composition is delivered. The one or more ports or orifices permit controlled placement of the adhesive composition to the bone fracture site.

In some embodiments, the cannula is constructed and arranged for one or more axes of motion, e.g., uni-/bi-/or multi-modal motion. In this configuration, the one or more axes of motion permit a broad range of motion of the cannula. For example, the motion may be deflection of the cannula tip. In some embodiments, the motion may include direction control of the full cannula. In some embodiments, the cannula tip further comprises a reinforcing guidewire, pull wire, or flat wire which aids in the steering or guidance of the cannula. The wire may be braided or coiled individual wires, variable picks-per-inch (PPI) longitudinal wires, or high tensile, non-metal fibers. In some embodiments, a coil wire reinforcement provides increased flexibility and kink resistance compared to a braided wire. In some embodiments, a pull wire as described herein may be used to steer or guide the cannula tip. For example, a single pull wire having a single direction of flexion in combination with the rotatability of the cannula device provides similar capabilities to multiple pull wires or axes of flexion. In some embodiments, the cannula comprises a variable durometer to permit curvature or bending of the cannula or cannula tip. In some embodiments, the cannula comprises a rigidity sufficient to penetrate and manipulate the soft tissue and viscera surrounding the wound site, e.g., bone fracture site. In some embodiments, any or all components of the delivery device relevant to registration of the cannula by the imaging system are sufficiently rigid. In some embodiments, the rigidity of the cannula permits clearing of debris from the wound site, e.g., bone fracture site, thus creating more space for delivery of an adhesive composition, e.g., therapeutic, bone regenerative and/or bioresorbable composition, and is constructed and arranged to withstand rotational or bending forces when displacing elements out of the wound site, e.g., bone fracture site, for preparation of the wound site. In some embodiments, a handle may be introduced by passing a trocar through the cannula to provide for manipulation of soft tissue and bone fragments at the wound site.

In some embodiments, an outer diameter of the cannula has a maximum diameter of 15 millimeters. In some embodiments, the outer diameter of the cannula is less than 15 millimeters. In some embodiments, the outer diameter of the cannula is less than 10 millimeters. In some embodiments, the outer diameter of the cannula is less than 5 millimeters. In some embodiments, an inner diameter of the cannula has a maximum diameter of 5 millimeters. In some embodiments, the inner diameter of the cannula is less than 5 millimeters, e.g., less than 4 mm, less than 3 mm, less than 2 mm, less than 1 mm, less than 0.5 mm, or less. In some embodiments, the inner diameter of the cannula is between 6 and 28 gauge (e.g., 6 to 14 gauge, 10 to 28 gauge, 16 to 24 gauge, or 18 to 22 gauge). In some embodiments, the inner diameter of the cannula is between about 0.05 mm to 5 mm, e.g., 1.5 mm to 4.5 mm, 0.1 mm to 2.75 mm, 0.1 mm to 1 mm or 0.4 mm to 1 mm. In some embodiments, the cannula for delivery of a therapeutic composition is made from a polymer.

In some embodiments, the delivery system may comprise a balloon or mesh containment device. In some embodiments, balloon or mesh containment device is expandable, resorbable, porous and/or microporous, flexible, biocompatible, and can be of various sizing. In some embodiments, the containment device is used to define the spatial limits for controlled delivery of an adhesive composition, e.g., a therapeutic composition, bone regenerative composition, and/or bioresorbable composition, to the wound site, e.g., bone fracture site. In some embodiments, the containment device is minimally invasive, e.g., delivered via percutaneous delivery from the cannula prior to delivery of the therapeutic composition. In some embodiments, the containment device is stored within the cannula where engagement of the delivery system delivers the balloon or mesh containment device to the surgical site. In some embodiments, the containment device is expandable as an adhesive composition is delivered out of the cannula tip into the containment device for controlled delivery. In some embodiments, microporosity (e.g., pores less than 250 μm, preferably less than 100 μm) of the balloon or mesh containment device may prevent unwanted bulk leakage of the adhesive composition, i.e., the pores of the containment device are sized to permit contact between the adhesive composition with the adjacent tissue, e.g., hard tissue, e.g., bone, at the wound site, e.g., bone fracture site, for therapeutic benefit, e.g., adhesion, bone regeneration, bioresorption, antimicrobial action, antibacterial action, and/or antifungal action, of the therapeutic composition are delivered. The balloon or mesh containment device may not prevent molecular or ionic diffusion, nor seal the contents of the containment device since there is microporosity (e.g., pores greater than 1 μm, preferably greater than at least 5 μm). In some embodiments, the balloon or mesh material is bioresorbable and its rate of resorption may be controlled by selection of the material, e.g., biopolymer. In some embodiments, the porosity of the balloon or mesh containment device may be anisotropic permitting the adhesive composition, e.g., therapeutic, bone regenerative and/or bioresorbable composition, to pass in specific directions, e.g., where multiple bone fragments are to be adhered together while restricting flow of the adhesive composition in other directions, e.g., where there is soft tissue or a joint articulation surface is to be avoided. In some embodiments, the balloon or mesh containment device is stored within the cannula in a folded condition to permit deployment and expansion within the wound site, e.g., bone fracture site and further permitting repeatable realignment of the anisotropic porosity. In some embodiments, the balloon contains one or more radiopaque elements to provide for image-guided control the balloon's alignment with an imaging modality, e.g., fluoroscopic, mechanical or computerized tomographic, or ultrasound imaging. In some embodiments, the balloon or mesh containment device may be detachable from the cannula following delivery of the adhesive composition. In some embodiments, detachment may be effectuated by exceeding a certain injection force (e.g., at least 100 N, preferably at least 200 N), by exceeding a certain cannula withdrawal force (e.g., at least 100 N, preferably at least 200 N), or by exceeding a certain cannula torque (e.g., at least 5 N/cm, preferably at least 10 N/cm) after the injected therapeutic composition reached a certain level of viscosity (at least 10,000 cP) to be able to resist the detachment force or torque. In some embodiments, detachment is aided by engineered weakness at the interface, or via cutting features or marks on the cannula tip.

In some embodiments, the delivery device may permit controllable delivery of the adhesive composition, e.g., therapeutic composition, bone regenerative composition, and/or bioresorbable composition, relative to a desired amount to be delivered. The amount of the adhesive composition delivered may be controlled to achieve a desired property of the cured adhesive composition at the wound site and/or a desired performance of the adhesive composition during delivery of the therapeutic composition. In some embodiments, delivering the adhesive composition further comprises delivering an amount sufficient to reduce or prevent leakage of the adhesive composition from the wound site. For example, the amount of therapeutic composition delivered may be controlled to be within a desired range, may be controlled to be above a minimum threshold amount, and/or may be controlled to be below a maximum threshold amount. In some embodiments, the method of delivery further comprises comparing the amount of the adhesive composition delivered to the wound site to a desired quantity of adhesive composition to be delivered to the wound site, e.g., via acquiring image data of the wound site.

In some embodiments, the device further includes one or more fiducial features (e.g., one or more fiducial features on the device and/or one or more fiducial features to be placed on or near the wound site, e.g., bone site) for telemetric referencing of the position and/or orientation of guiding structures of the device. In some embodiments, the delivery device incorporates one or more fiducial features for telemetric referencing of the position and/or orientation of guiding structures of the delivery device for guidance in application of a adhesive composition, e.g., therapeutic composition, bone regenerative composition, and/or bioresorbable composition. In some embodiments, the one or more fiducial features may be used by computer guidance software for the application of the adhesive composition. In some embodiments, the computer guidance software provides an indication, e.g., to a user or operator, that a desired or target location for application of the adhesive composition has been reached. In some embodiments, a virtual plan for the procedure may be established, allowing the computer guiding software to control the timing, direction, volume, or rate of the application of the adhesive composition directly. In some embodiments, under preestablished parameters, the guiding software uses real time or near real time data from the image data acquisition system, e.g., fluoroscopic imaging system, tomographic or ultrasound or x-ray imaging system, to control the timing, direction, volume, or rate of application of the adhesive composition.

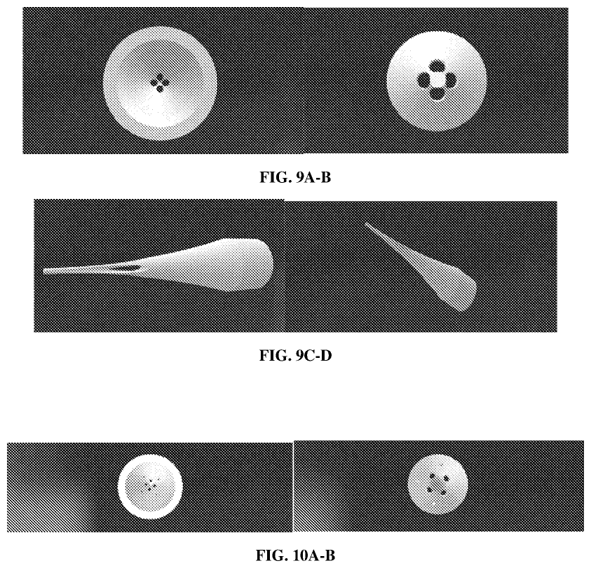

In some embodiments, the delivery device is constructed and arranged to direct the flow of the adhesive composition at an angle relative to the delivery device's long axis, or concentrically and parallel to its axis, e.g., laterally at a previously determined angle or straight out the tip of the cannula. In some embodiments, the delivery device is constructed and arranged to direct the flow of the adhesive composition over a range of radial angles, e.g., radially narrow or creating an angular spread of the therapeutic composition. For example, a distal portion of a cannula tip may comprise multiple ports or channels permit redirecting or guiding the adhesive composition, e.g., therapeutic composition, bone regenerative composition and/or bioresorbable composition, to the wound site. FIGS. 9A-9D through FIGS. 12A-12B depict multiple views of various cannula tips. In some embodiments, the delivery device is constructed and arranged for rotational and translational movement. The present disclosure further provides for cannula tips which may include an image guidance registration modality, e.g., software processing parameter, or fiducial feature (e.g., a cannula tip with an image contrast agent, e.g., BaSO₄) permitting visualization of the cannula tip. In some embodiments, the registration modality is disposed near the cannula tip, e.g., located on or adjacent to a component of the delivery device. In this configuration, the wound site can be imaged before delivering the therapeutic composition permitting repositioning of the delivery device and for guiding the trajectory of the adhesive composition prior to delivery. Such control may prevent delivery overflow and spillage of the adhesive composition, e.g., therapeutic composition, bone regenerative composition and/or bioresorbable composition, out of the wound site. In some embodiments, the software processing parameter(s) of the delivery device is/are generated by at least one processor, which is/are on the delivery device itself, a controller, or another component, such as another component of the delivery system.

In some embodiments, the delivery device may comprise a navigational registration system, e.g., on the shaft or body of the delivery system or part of the cannula tip, permitting imaging, e.g., imaging via fluoroscopic, tomographic, or ultrasonic imaging. An example is illustrated in FIG. 2. In some embodiments, an outline of the delivery device, including the tip of the delivery device, e.g., cannula, can be visualized with respect to the patient's tissue, e.g., wound site. In some embodiments, the projection and registration of the delivery device with the imaging system is based on a computer aided design file that is registered for the delivery device based on the registration element located on the delivery device.

In some embodiments, the delivery device may be controlled by a mechanical placement device, e.g., a remote controlled or robotic device. In some embodiments, a depth, angle of insertion, and rotation about one or more axes of the delivery device may be predetermine or preprogrammed.

In some embodiments, delivery of the adhesive composition, e.g., therapeutic composition, bone regenerative composition, and/or bioresorbable composition, to the wound site is controlled during a procedure. In some embodiments, control of delivery of the adhesive composition is achieved by use of a controller in communication with one or more actuators. In some embodiments, the controller includes one or more processors constructed and arranged to generate a signal delivered to the one or more actuators to perform one or more of: initialize delivery of the adhesive composition to the site, stop delivery of the adhesive composition to the site, and/or modulate delivery of the adhesive composition to the site, e.g., in substantially real time. In some embodiments, the controller transmits a signal to the one or more actuators to deliver the adhesive composition. In some embodiments, the controller is in communication with one or more sensors and is configured transmit a signal to the one or more actuators based on a signal received by the controller from one or more sensors. In some embodiments, controlling delivery of the adhesive composition comprises controlling one or more of: an amount of therapeutic composition delivered, a location of delivery of the adhesive composition, a duration of delivery of the adhesive composition, and/or a flow rate of delivery of the adhesive composition.

In some embodiments, the controller includes at least one processor and at least one memory component. The at least one memory component may include machine-readable instructions (e.g., instructions in the form of one or more algorithms) stored in the memory component to be executed by the at least one processor. In some embodiments, the at least one processor is configured to execute the instructions to cause the controller to transmit (e.g., via a wired or wireless connection) one or more signals to the delivery device and/or an imaging device. In some embodiments, the at least one processor may be configured to execute the machine-readable instructions stored on the memory component to send one or more signals to the delivery device to control parameters of delivery of the therapeutic composition to the wound site. For example, the one or more signals transmitted to the delivery device may cause the delivery device (e.g., via actuators of the delivery device) to start, stop, and/or modulate flow of the adhesive composition to the site, and/or may cause the delivery device to direct a flow of the adhesive composition to a different location, at a different angle, and/or in a different pattern. In some embodiments, the signals generated by the controller are based on one or more signals received from the image acquisition unit and/or the delivery device.

### Visualization

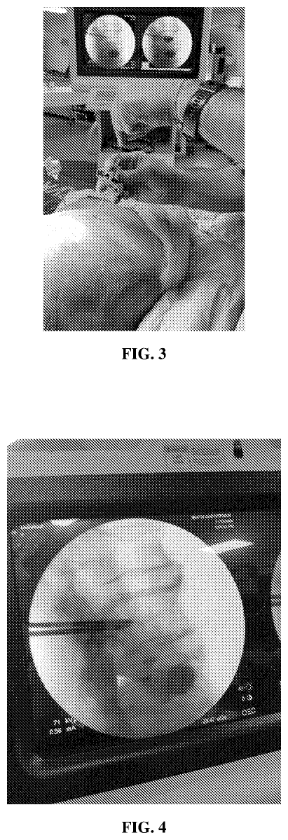

The present disclosure further provides systems and methods of visualization, e.g., fluoroscopy, mechanical or computerized tomography, ultrasound or X-ray imaging, of the site, e.g., wound site, e.g., bone site. In some embodiments, visualization of the site includes acquiring image data of the site. In some embodiments, image data is acquired via fluoroscopic imaging, mechanic tomography or computerized tomography, ultrasound imaging, x-ray imaging, or another suitable method of image data acquisition. In some embodiments, visualization of the site may include visualization of one or more anatomical structures at the site, e.g., a bone fracture, bone fragments and/or of a adhesive composition, e.g., therapeutic, bone regenerative and/or bioresorbable composition, being delivered to the site. In some embodiments, visualization of the one or more anatomical structure(s) at the wound site and/or the adhesive composition may include visualization of an agent, e.g., a contrast agent, present in the therapeutic composition or on the delivery device. Visualization of the site may include static or still images or video sequences. In some embodiments, visualization includes substantially real time visualization. In some embodiments, static or still images may be acquired at predetermined intervals, e.g., every 2 seconds or every 5 seconds. In some embodiments, visualization is performed prior to a procedure. FIG. 3 depicts a fluoroscopic imaging machine used to perform percutaneous delivery of a adhesive composition into a vertebral space during a kyphoplasty procedure. In some embodiments, fluoroscopic images trace an imaging or contrast agent, e.g., barium sulfate, present in the adhesive composition. Use of image guided delivery, e.g., fluoroscopic imaging, may permit proper placement and delivery of an effective amount of the adhesive composition and further may prevent overflow of the adhesive composition out of the desired site, e.g., out of the vertebral space as in FIG. 3 or FIG. 4. which illustrate zoomed in views of fluoroscopic imaging guidance used to assist a surgeon in delivering a composition, e.g., an adhesive composition, into a vertebral space during a kyphoplasty procedure.

In some embodiments, visualization comprises acquiring image data, processing the acquired image data to provide an image output, and displaying the resulting image output to a visualization device, e.g., fluoroscopic imaging device, e.g., the Philips BV29 Fluoro C-Arm, as one or both of static or still images or video sequences. Acquisition of image data, e.g., fluoroscopic imaging, may occur prior to, during, or after delivery of the adhesive composition, e.g., therapeutic composition, bone regenerative composition, and/or bioresorbable composition. In some embodiments, images are acquired during placement of the adhesive composition, following, e.g., within a short duration after, placement of the adhesive composition, and a longer period of time, e.g., 1 week, 2 weeks, 3 weeks, longer, following of the adhesive composition. In some embodiments, the image data of the wound site is acquired prior to positioning the delivery device for delivery of the adhesive composition. In some embodiments, the image data of the wound site may be acquired prior to delivering the adhesive composition to the wound site following positioning of the delivery device.

In some embodiments, fluoroscopic imaging prior to delivery of the adhesive composition permits visualization of the wound site and the location of the cannula tip within a patient. In some embodiments, imaging during delivery of the adhesive composition permits confirmation of the delivery of the adhesive composition. In some embodiments, imaging during delivery of the adhesive composition may be performed on timescales of clinical relevance, e.g., on the order of seconds, e.g., every 2 seconds or every 4 seconds. In some embodiments, fluoroscopic imaging at an interval following delivery of the adhesive composition permits monitoring of the curing of the adhesive composition and healing of the surrounding wound site, e.g., bone healing and regeneration. Multiple images at each time interval, e.g., from different angles or via multiple modes of imaging, e.g., fluoroscopic or tomographic imaging and ultrasound imaging, may be collected. In some embodiments, the point of view or perspective of the imaging device, e.g., fluoroscopic imaging device, is fixed during image acquisition. In some embodiments, the point of view or perspective is moved or adjusted. In some embodiments, two or more points of view or perspectives are used substantially simultaneously to capture multiple angles of the field of interest without requiring movement of the imaging device or the patient. In some embodiments, the multiple captured angles are combined to form a 3D rendering of the wound site. In some embodiments, the 3D rendering of the wound site is created in substantially real time to aid in delivery of the adhesive composition to the wound site.

In some embodiments, the imaging device collects image data using one or more wavelengths of visible light, one or more wavelengths of ultraviolet light, one or more wavelengths of infrared light, one or more wavelengths of X-rays, or one or more other clinically relevant electromagnetic wavelengths. In some embodiments, acquiring image data comprises acquiring data related to heat, position of sound waves, position of X-rays, position of radio waves, and/or the presence of an agent (e.g., a contrast agent or radioisotope).

In some embodiments, the imaging device is a fluoroscopy machine, an X-ray device, an optical camera, a mechanical or a computerized tomography device, or another imaging device. In some embodiments, the imaging device relies on other imaging technology. In some embodiments, the imaging device is an ultrasound device. In some embodiments, the imaging device is a computed tomography (CT) scanner. In some embodiments, the imaging device is a magnetic resonance imaging (MRI) machine. Any of these imaging devices selected may come with a corresponding visualization mechanism. In some embodiments, acquiring the image data comprises use of a computer-assisted imaging system. In some embodiments, use of a computer-assisted imaging system comprises acquiring signal data from a wound site, converting the signal data to image data, and/or displaying the image data to a user.

In some embodiments, the imaging device is configured to detect a marker. In some embodiments, the marker is generated by presence of a contrast agent. In some embodiments, the contrast agent comprises barium (e.g., BaSO₄), iodine (e.g., iohexol, iodioxanol, ioversol, iothalamate, iosimenol, iopamidol, iopromide, or ioxaglate), bismuth (e.g., bismuth oxide), or gadolinium. In some embodiments, the contrast agent is formulated as a liposome. In other embodiments, the contrast agent is a gas (e.g., carbon dioxide, nitrogen, air, etc.). In some embodiments, the marker is a radiocontrast agent. In some embodiments, the marker is a radiopaque contrast agent.

In some embodiments, image data is represented by values in a database, by values assigned to cells in an array, or by values assigned to voxels in an array. In some embodiments, acquiring image data comprises acquiring a plurality of two-dimensional plane images, such as a stack or a series of two-dimensional plane images. In some embodiments, the two-dimensional plane images are generated simultaneously and presented on a display. In some embodiments, two-dimensional plane images for more than one view are presented on the display in substantially real time. In some embodiments, control of the dispensing of the adhesive composition is based on image data from more than one view of the site in substantially real time. In some embodiments, the image data is displayed to the user on a display located in the same location as the patient. In some embodiments, the image data is displayed to the user on a display located in a different location than the patient.

In some embodiments, acquiring image data comprises operating an endoscope camera. In some embodiments, acquiring image data using the endoscope camera comprises use of the endoscope camera as a probe in a lumen at the wound site. In some embodiments the endoscopic camera is continuously operated during a procedure. In some embodiments, the endoscopic camera may be operated during intermittently, e.g., during one or more intervals, during the procedure. In some embodiments, the endoscope camera is operated to provide substantially real time three-dimensional views of the wound site and/or of tissue near the wound site. In some embodiment, the endoscope camera is operated to provide substantially real time views of the wound site and still images may be taken at predetermined time intervals on the order of seconds, e.g., 1 second apart, 2 seconds or 3 seconds apart.

In some embodiments, the system, method, and/or apparatus for acquiring image data is a system, method and/or apparatus described in U.S. Pat. No. 10,070,828 or International Patent Application Publication WO 2019/060843, each of which is incorporated herein by reference in its entirety.

In some embodiments, the system, method, and/or apparatus for acquiring image data may be a system described in U.S. Pat. No. 10,070,828. In some embodiments, the system, method, and/or apparatus comprises at least one radiation source configured to move along at least one closed path; at least one detector configured to receive radiation from the at least one radiation source as the at least one radiation source is moved along the at least one closed path to allow for generating reconstruction image data of at least a portion of a three-dimensional object; and a gantry configured to enclose the at least one radiation source within an enclosed portion of the gantry, wherein the gantry is constructed and arranged to not include exposed moving parts during an imaging process using the imaging system. In some embodiments, the gantry is further configured to enclose the at least one radiation source without fully enclosing the three-dimensional object. In some embodiments, the at least one radiation source and the at least one radiation detector are positioned in the imaging system such that at least a portion of a three-dimensional object can be positioned in between the at least one radiation source and the at least one x-ray radiation detector for image acquisition. In some embodiments, the at least one radiation detector is spaced apart from the at least one closed path. In some embodiments, the reconstruction image data comprises three-dimensional reconstruction image data. In some embodiments, the at least one radiation source is constructed and arranged to move continuously while emitting radiation. In certain embodiments, the at least one radiation source is configured to move along the at least one closed path with respect to the at least one radiation detector.

In some embodiments, the system and/or apparatus for acquiring image data includes a processor configured to receive image data from the at least one radiation detector and apply a reconstruction algorithm to generate a reconstructed 3D image of the three-dimensional object. In some embodiments, the imaging system is configured to provide real-time or near real-time imaging by evolving a reference image of at least a portion of the three-dimensional object to form a sequence of 3D volumes as a function of time, where the reference image is a preceding reconstructed 3D image which is updated at least once over time, to generate the three-dimensional reconstruction image data; and an interface to provide the three-dimensional reconstruction image data to a user

In some embodiments, the system, method, and/or apparatus for acquiring image data is a system described in International Patent Publication WO 2019/060843. In some embodiments, the system, method, and/or apparatus for acquiring image data comprises an imaging modality configured to generate an image dataset for a target object; and at least one memory device including machine readable instructions stored thereon that, when executed by at least one processor, cause the imaging system to reconstruct an image of the target object using an iterative reconstruction technique that includes a machine learning model as a regulizer to reconstruct the image of the target object. In some embodiments, the machine learning model is trained, prior to generating the image dataset, to define object features and/or remove reconstruction artifacts using learning datasets that include image data related to the target object to be imaged using the imaging modality. In some embodiments, the machine learning model is included in the iterative reconstruction technique to introduce the object features into and/or remove reconstruction artifacts from the image of the target object being reconstructed.

### Composition

The present disclosure provides that the composition delivered to the wound site is an adhesive composition, e.g., a composition having therapeutic properties such as soft and hard tissue adhesion, biocompatibility, bone regeneration, and bioresorption. In some embodiments, the therapeutic properties of the adhesive composition further comprise antimicrobial behavior, antibacterial behavior, antifungal behavior, and other preferential properties. In some embodiments, the adhesive composition, e.g., therapeutic, bone regenerative and/or bioresorbable composition, comprises a multivalent metal salt, an organic compound, and an aqueous medium. In some embodiments, the multivalent metal salt comprises tetracalcium phosphate or tricalcium phosphate (e.g., α-tricalcium phosphate or β-tricalcium phosphate). In some embodiments, the therapeutic composition additionally comprises a contrast agent and/or an additive. In some embodiments, the therapeutic composition described herein includes any therapeutic composition disclosed in U.S. Pat. No. 8,765,189, which is incorporated herein by reference its entirety.

In some embodiments, the adhesive composition is self-setting and resorbable so as to be replaced by native bone. For example, the interaction of the components of the adhesive composition may result in the production of a tacky and adhesive mixture. In particular, the interaction may result in a viscous substance which then solidifies forming a bond to high-energy surfaces, e.g., bone, metal, or glass. In some embodiments, the adhesive bond interaction between the adhesive composition and the substrate surface occurs if the substrate is dry or wet (e.g., dampened or submerged in an aqueous medium).

In some embodiments, the adhesive composition has an adhesive effect to cause bone regeneration, e.g., is osteostimulative, osteopromotive or osteoinductive. In some embodiments, the adhesive composition comprises a porosity and stiffness matching that of the native bone when cured, thereby preventing stress shielding and subsidence.

In some embodiments, the adhesive composition, e.g., therapeutic, bone regenerative and/or bioresorbable composition, further comprises a contrast agent (e.g., a radiopaque agent or radioisotope). In some embodiments, the contrast agent comprises barium (e.g., BaSO₄), iodine (e.g., iohexol, iodioxanol, ioversol, iothalamate, iosimenol, iopamidol, iopromide, or ioxaglate), bismuth (e.g., bismuth oxide), or gadolinium. In some embodiments, the contrast agent is formulated as a liposome. In some embodiments, the contrast agent is a gas (e.g., carbon dioxide, hydrogen, or air). In some embodiments, the amount of the agent in the adhesive composition is less than about 20%, 15%, 10%, 9%, 8%, 7%, 6%, 5%, 4%, 3%, 2%, 1%, 0.5%, 0.1%, 0.05%, or 0.01% (w/w or w/v) of the total adhesive composition. In some embodiments, the adhesive composition has an adhesive strength upon curing of about 250 kPa or more, e.g., about 100 kPa to about 12,000 kPa, depending on the application and the particular components and ratios of components in said adhesive compositions. In some embodiments, the adhesive strength of the adhesive compositions in the cement-like state is between about 100 kPa and e.g., about 10,000 kPa, about 9,000 kPa, about 8,000 kPa, about 7,000 kPa, about 6,000 kPa, about 5,000 kPa, about 4,000 kPa, about 3,000 kPa, about 2,000 kPa, about 1,000 kPa, about 750 kPa, about 500 kPa, about 250 kPa, or about 200 kPa. In some embodiments, the adhesive strength of the adhesive compositions in the cement-like state is between about 100 kPa, about 200 kPa, about 300 kPa, about 400 kPa, about 500 kPa, about 600 kPa, about 700 kPa, about 800 kPa, about 900 kPa, about 1,000 kPa, about 2,500 kPa, about 5,000 kPa, about 7,500 kPa, about 10,000 kPa or about 12,000 kPa. In some embodiments, the adhesive strength of the adhesive compositions in the cement-like state is in the range of about 200 kPa and about 2,500 kPa. In some embodiments, the adhesive strength of the adhesive compositions in the cement-like state is greater than 100 kPa.

Adhesive compositions of the present disclosure address many of the known shortcomings of currently available bone cements. Currently available calcium phosphate bone cements are typically only void fillers without the ability to chemically adhere to bone and do not have therapeutic properties, such as being osteostimulative, osteopromotive or osteoinductive. The adhesive compositions described herein may have a sufficiently high viscosity that they can be directed by injection though a delivery system but maintain their cohesion as force is applied from the delivery system, such that the liquid and solid components of the adhesive composition do not separate or migrate from the site, i.e., cement extravasation. The higher viscosity and cohesion of the adhesive composition mitigates the risks of extravasation or leakage of the adhesive composition upon application, increasing patient safety.

In some embodiments, the dry components of the adhesive composition are stored in a chamber of a second vessel of a delivery system and the aqueous medium is stored within a first vessel of a delivery system. In some embodiments, the adhesive composition comprises: a multivalent metal salt; a e.g., a compound (e.g., a compound of Formula (I), Formula (II), Formula (III), Formula (IV), Formula (V), Formula (VI), or a combination thereof), an aqueous medium; and a contrast agent.

In some embodiments the multivalent metal salt comprises one or more alkaline earth metals or metalloids, e.g., beryllium, magnesium, barium, radium, strontium, silicon, aluminum, or calcium. In some embodiments, the multivalent metal salt may comprise calcium phosphate, calcium nitrate, calcium citrate, calcium carbonate, magnesium phosphates, sodium silicates, titanium phosphates, strontium phosphates, barium phosphates, zinc phosphates, calcium oxide, magnesium oxide, calcium silicate, calcium aluminate, zinc aluminate, and combinations thereof. In some embodiments, the multivalent metal salt may comprise tetracalcium phosphate or alpha-tricalcium phosphate. In some embodiments, the multivalent metal salt is tetracalcium phosphate.

In some embodiments, the organic compound is a compound of Formula (I):

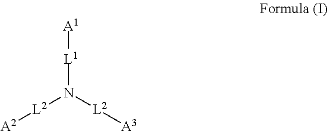

or a salt thereof, wherein:
each of A¹, A², and A³ is independently selected from an acidic group (e.g., a carboxyl or phosphonyl); and
each of L¹, L², and L³ is independently bond, alkylene (e.g., C₁-C₆ alkylene), or heteroalkylene (e.g., C₁-C₆ heteroalkylene).

In some embodiments, each of A¹, A², and A³ is independently a carboxyl or phosphonyl. In some embodiments, A¹ is carboxyl, and A² and A³ are phosphonyl. In some embodiments, A¹, A² and A³ are phosphonyl.

In some embodiments, each of L¹, L², and L³ is C₁-C₃ alkylene. In some embodiments, each of L¹, L², and L³ is a C₁alkylene.

In some embodiments, the compound of Formula (I) is a compound of Formula (I-a) or (I-b):

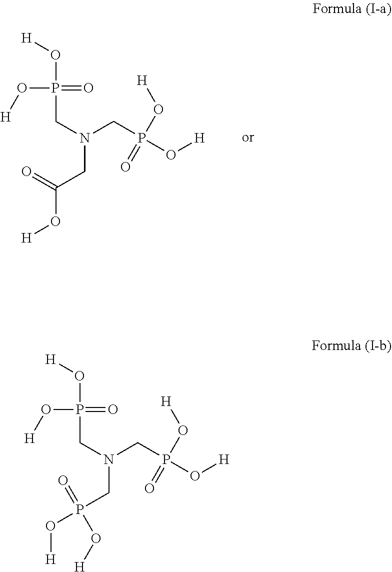 [SMILES: O=C(O)CN(CP(=O)(O)O)CP(=O)(O)O ; O=P(O)(O)CN(CP(=O)(O)O)CP(=O)(O)O]

In some embodiments, the aqueous medium is water. In some embodiments, the adhesive composition further comprises an additive.

In some embodiments, the organic compound is a compound of Formula (II) is:

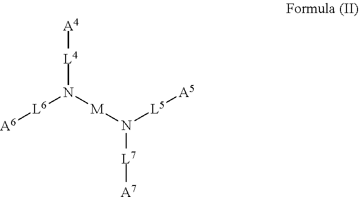

or a salt thereof, wherein:
each of A⁴, A⁵, and A⁶, is independently selected from an acidic group (e.g., a carboxyl or phosphonyl);
A⁷ is selected from an acidic group (e.g., a carboxyl or phosphonyl), a hydrogen atom, an alkyl, an aryl, a hydroxy group, a thio group, and an amino group;
each of L⁴, L⁵, L⁶, and L⁷ is independently bond, alkylene (e.g., C₁-C₆ alkylene), or heteroalkylene (e.g., C₁-C₆ heteroalkylene); and
M is alkylene (e.g., C₁-C₆ alkylene) or heteroalkylene (e.g., C₁-C₆ heteroalkylene).

In some embodiments, A⁴, A⁵, A⁶ and A⁷ are carboxyl.

In some embodiments, L⁴, L⁵, L⁶, and L⁷ are C₁-C₃ alkylene. In some embodiments, L⁴, L⁵, L⁶, and L⁷ are C₁ alkylene.

In some embodiments, M is C₁-C₄ alkylene. In some embodiments, M is C₂ alkylene. In some embodiments, M is C₃ alkylene. In some embodiments, M is C₁-C₆ heteroalkylene. In some embodiments, M is C₆ heteroalkylene. In some embodiments, M is bis(ethyleneoxy)ethylene. In some embodiments, M includes side chains. In some embodiments, M includes multiple side chains. In some embodiments, M includes one or multiple carboxymethylene side chains. In some embodiments, M includes one or multiple N-carboxymethylene groups or N-hydroxymethylene groups.

In some embodiments, the compound of Formula (II) includes three, four, five, six, or more N-carboxymethylene groups. In some embodiments, the compound of Formula (II) comprises ethylenediamine tetraacetic acid (EDTA). In some embodiments, the compound of Formula (II) is a compound of Formula (II-a), (II-b), (II-c), (II-d), (II-e), or (II-f):

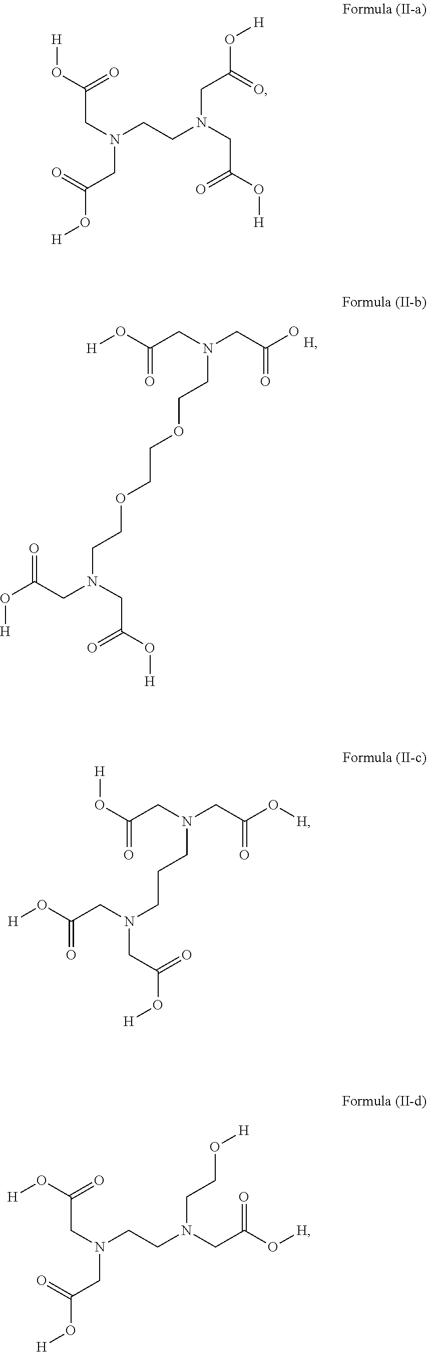

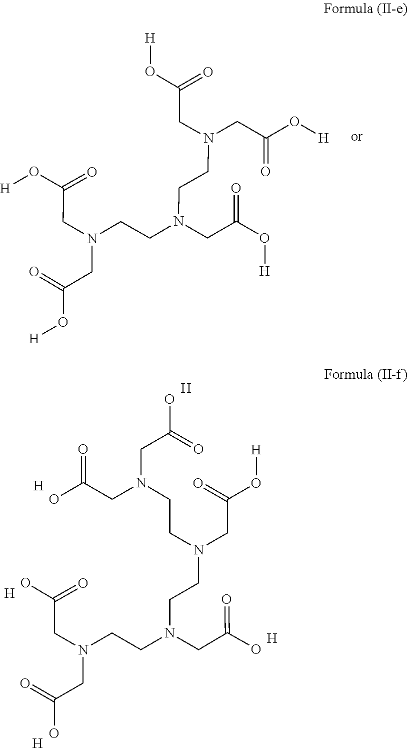

In some embodiments, the aqueous medium is water. In some embodiments, the adhesive composition further comprises an additive.

In some embodiments, the organic compound is a compound of Formula (III) is:

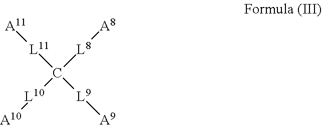

or a salt thereof, wherein:
each of A⁸ and A⁹ is independently selected from an acidic group (e.g., a carboxyl or phosphonyl);
each of A¹⁰ and A¹¹ is independently selected from an acidic group (e.g., a carboxyl or phosphonyl), a hydrogen atom, an alkyl, aryl, a hydroxy group, a thio group, and an amino group;
each of L⁸, L⁹, L¹⁰ and
L¹¹ is independently bond, alkylene (e.g., C₁-C₆ alkylene), or heteroalkylene (e.g., C₁-C₆ heteroalkylene.

In some embodiments, A⁸, A⁹, and A¹⁰ are carboxyl. In some embodiments, A¹⁰, A¹¹, are a hydrogen atom. In some embodiments, A¹¹ is a hydroxy or amino group. In some embodiments, L⁸, L⁹, L¹⁰, and L¹¹ are a bond. In some embodiments, L⁸ and L⁹ are C₁-C₃ alkylene. In some embodiments L¹¹ is a heteroalkylene (e.g., C₁-C₆ heteroalkylene). In some embodiments L¹¹ is methylenethiomethylene. In some embodiments, the compound of Formula (III) comprises citric acid or malonic acid. In some embodiments, the compound of Formula (III) is a compound of Formula (III-a), (III-b), (III-c), or (III-d):

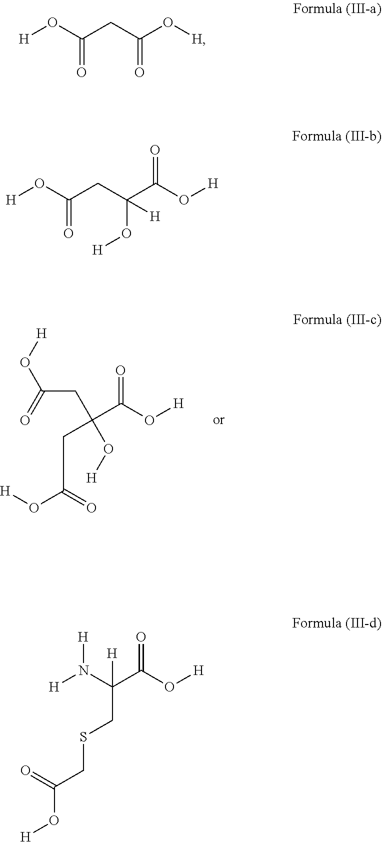

In some embodiments, the aqueous medium is water. In some embodiments, the adhesive composition further comprises an additive.

In some embodiments, the organic compound is a compound of Formula (IV):

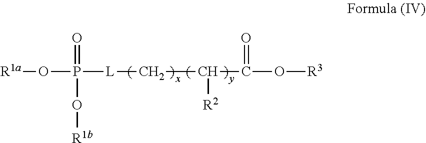

or a salt thereof, wherein:
L is O, S, NH, or CH₂;
each of R^{1a} and R^{1b} is independently H, an optionally substituted alkyl, or an optionally substituted aryl;
R² is H, NR^{4a}R^{4b}, C(O)R⁵, or C(O)OR⁵;
R³ is H, an optionally substituted alkyl, or an optionally substituted aryl;
each of R^{4a} and R^{4b} is independently H, C(O)R⁶, or an optionally substituted alkyl;
R⁵ is H, an optionally substituted alkyl, or an optionally substituted aryl; R⁶ is an optionally substituted alkyl or an optionally substituted aryl; and
each of x and y is independently 0, 1, 2, or 3.

In some embodiments, L is O or S. In some embodiments, L is O. In some embodiments, each of R^{1a} and R^{1b} is independently H. In some embodiments, L is O, and each of R^{1a} and R^{1b} is H.

In some embodiments, R² is selected from H, NR^{4a}R^{4b}, and C(O)R⁵. In some embodiments, R² is NR^{4a}R^{4b}. In some embodiments, R² is NR^{4a}R^{4b} and each of R^{4a} and R^{4b} is independently H.

In some embodiments, L is O, each of R^{1a} and R^{1b} is independently H, R² is NR^{4a}R^{4b}, and each of R^{4a} and R^{4b} is independently H.

In some embodiments, R³ is H. In some embodiments, L is O, each of R^{1a} and R^{1b} is independently H, R² is NR^{4a}R^{4b}, each of R^{4a} and R^{4b} is independently H, and R³ is H.

In some embodiments, each of x and y is independently 0 or 1. In some embodiments, each of x and y is independently 1. In some embodiments, L is O, each of R^{1a} and R^{1b} is independently H, R² is NR^{4a}R^{4b}, each of R^{4a} and R^{4b} is independently H, R³ is H, and each of x and y is 1.

In some embodiments, the compound of Formula (IV) is phosphoserine. In some embodiments, the aqueous medium is water.

In some embodiments, the organic compound is a compound of Formula (V) is:

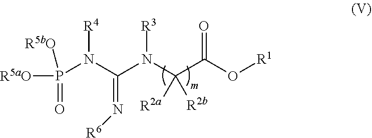

or a salt thereof, wherein:
R¹ is H, optionally substituted alkyl, optionally substituted alkenyl, optionally substituted alkynyl, optionally substituted aryl, or optionally substituted heteroaryl;
each of R^{2a} and R^{2b} is independently H, optionally substituted alkyl, hydroxy, alkoxy, or halo;
each of R³ and R⁴ is independently H or optionally substituted alkyl;
each of R^{5a} and R^{5b} is independently H, optionally substituted alkyl, optionally substituted alkenyl, optionally substituted alkynyl, optionally substituted aryl, or optionally substituted heteroaryl;
R⁶ is H or optionally substituted alkyl; and
m is 1, 2, 3, 4, or 5.

In some embodiments, R¹ is H. In some embodiments, each of R^{2a} and R^{2b} is independently H. In some embodiments, m is 1. In some embodiments, each of R³ and R⁴ is H. In some embodiments, each of R^{5a} and R^{5b} is independently H. In some embodiments, R⁶ is H. In some embodiments, the compound of Formula (V) is a phosphocreatine. In some embodiments, the compound of Formula (V) is Formula (V-a):

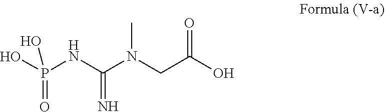

In some embodiments, the compound of Formula (V) is phosphocreatine (e.g., Formula (V-a). In some embodiments, the aqueous medium is water.

In some embodiments, the organic compound is a compound of Formula (VI) is:

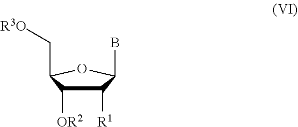

or a salt thereof, wherein:
B is a nucleobase;
R¹ is H, OR⁴, or halo;
R² is H, optionally substituted alkyl, optionally substituted alkenyl, optionally substituted alkynyl, optionally substituted aryl, optionally substituted heteroaryl, optionally substituted cycloalkyl, or optionally substituted heterocyclyl;
R³ is H, optionally substituted alkyl, or a phosphate moiety (e.g., monophosphate or diphosphate); and
R⁴ is H, optionally substituted alkyl, optionally substituted alkenyl, optionally substituted alkynyl, optionally substituted aryl, optionally substituted heteroaryl, optionally substituted cycloalkyl, or optionally substituted heterocyclyl.

In some embodiments, B is a naturally occurring nucleobase or a non-naturally occurring nucleobase. In some embodiments, B comprises adenine, cytosine, guanosine, thymine, or uracil. In some embodiments, each of R¹, R², and R³ is H. In some embodiments, R³ is a phosphate group, e.g., a monophosphate, diphosphate, or triphosphate. In some embodiments, the compound of Formula (VI) is Formula (VI-a) or (VI-b):

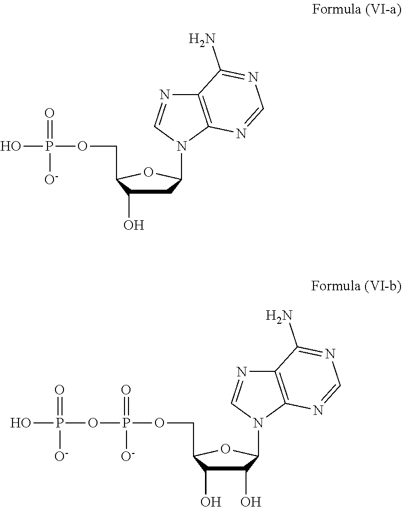

For example, the compound of Formula (VI) is 2′-deoxyadenosine monophosphate or 2′-deoxyadenosine diphosphate. In some embodiments, the aqueous medium is water.

In some embodiments, the aqueous medium comprises water (e.g., sterile water), saliva, buffers (e.g., sodium phosphate, potassium phosphate, sodium hydroxide, or saline (e.g., phosphate buffered saline)), blood, blood-based solutions (e.g., plasma, serum, bone marrow), spinal fluid, dental pulp, cell-based solutions (e.g., solutions comprising fibroblasts, osteoblasts, platelets, odontoblasts, stem cells (e.g., mesenchymal stem cells) histiocytes, macrophages, mast cells, or plasma cells), or combinations thereof in the form of aqueous solutions, suspensions, and colloids. In some embodiments, the aqueous medium comprises sterile water, distilled water, deionized water, sea water, or fresh water.

In an embodiment, the aqueous medium comprises a compound, e.g., a compound (e.g., a compound of Formula (I), Formula (II), Formula (III), Formula (IV), Formula (V), Formula (VI), or a combination thereof), in suspension in aqueous medium. This suspension of the compound (a compound (e.g., a compound of Formula (I), Formula (II), Formula (III), Formula (IV), Formula (V), Formula (VI), or a combination thereof) may be contained in a second chamber in the main body of the device.

In some embodiments, the dry components of the composition are provided as a premixed powder composition. These powders may exhibit a mean particle size of about 0.001 to about 1 mm, e.g., about 0.001 to about 0.25 mm, about 0.005 to about 0.15 mm, about 0.25 to about 0.75 mm, 0.25 to about 0.5 mm, 0.1 to about 0.05 mm, about 0.015 to about 0.025 mm, about 0.02 to about 0.06 mm, about 0.02 to about 0.04 mm, about 0.04 to about 0.1 mm, about 0.04 to about 0.06 mm, about 0.06 to about 0.15 mm, or about 0.06 to about 0.125 mm. In some embodiments, the powder has a mean particle size of less than about 1 mm. In some embodiments, the powdered components may be provided in a condensed pelletized version. In an embodiment, the particle size distribution is multi-modal to include any combination of mean particle sizes as described herein.

In another embodiment, the dry components are provided as granules. The granules may exhibit a mean granule size of about 0.05 mm to about 5 mm, e.g., about 0.1 to about 1.5 mm, about 0.125 to 1 mm, 0.125 to 0.5 mm, about 0.125 to 0.25 mm, about 0.25 to 0.75 mm, about 0.25 to 0.5 mm, about 0.5 to 1 mm, or about 0.5 to 0.75 mm. The granule size distribution may be multi-modal to include any combination of mean granule sizes as described herein. In some embodiments, the granules are porous, e.g., having a plurality of internal pores. The plurality of internal pores may be in fluid communication. The plurality of internal pores may be in fluid communication with the granule surface. In some embodiments, the plurality of internal pores are not in fluid communication with each other. In some embodiments, the pores are not in fluid communication with the granule surface.

In some embodiments, the components of the adhesive composition, e.g., therapeutic, bone regenerative and/or bioresorbable composition, further comprise an additive. Exemplary additives may include salts (e.g., calcium carbonate, calcium bicarbonate, sodium carbonate, sodium bicarbonate, sodium chloride, potassium chloride), fillers, formulation bases, viscosity modifiers (e.g., polyols (e.g., glycerol, mannitol, sorbitol, trehalose, lactose, glucose, fructose, or sucrose)), abrasives (e.g., bone fragments), coloring agents (e.g., dyes, pigments, or opacifiers), flavoring agents (e.g., sweeteners), medications that act locally (e.g., anesthetics, coagulants, clotting factors, chemotactic agents, and agents inducing phenotypic change in local cells or tissues), medications that act systemically (e.g., analgesics, anticoagulants, hormones, enzyme co-factors, vitamins, pain relievers, anti-inflammatory agents, chemotactic agents, or agents inducing phenotypic change in local cells or tissues), antimicrobial agents (e.g., antibacterial, antiviral, or antifungal agents) or combinations thereof. In some embodiments, the additive comprises a solidified formed of the adhesive composition in the form of granules of fibers, described in more detail below. In some embodiments, the additive comprises a polymer. Exemplary polymers include poly(L-lactide), poly(D,L-lactide), polyglycolide, poly(caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone -co-lactide-co-trimethylene-carbonate), poly(hydroxybutyrate-cohydroxyvalerate), poly(methylmethacrylate), poly(acrylate), polyamines, polyamides, polyimidazoles, poly(vinyl-pyrrolidone), collagen, silk, chitosan, hyaluronic acid, gelatin and/or mixtures thereof.

In some embodiments, the additive comprises calcium carbonate particles. In some embodiments, the calcium carbonate particles are nanoparticles and/or microparticles, e.g., having a diameter of about 10 nm to about 1000 nm to micrometer range (e.g., about 10 μm to about 1000 μm) as a porogen agent, e.g., to increase porosity during the setting reaction of the adhesive composition. In some embodiments, the additive comprises phospho(enol)pyruvic acid or phosphocreatine. In some embodiments, the additive comprises a nucleic acid or nucleotide. In some embodiments, the additive comprises a silicate or a phosphorylated amino acid.

In addition, copolymers of the above homopolymers may also be used. The general structural nature of a polymer (e.g., a polymer used as an additive in a composition (e.g., adhesive composition) described herein) may include a linear homo and copolymer, a cross linked polymer, a block polymer, a branched polymer, a hyper branched polymer, or a star shaped polymer. The polymers may be added to the formulation in the form of a solution, powder, fiber, resin, liquid crystal, hydrogel, chip, flake, granule, and the like. In some embodiments, the polymer additive is poly-lactic-glycolic acid (PLGA). In some embodiments, additives may be provided as powders or granules. In some embodiments, said powders may exhibit a mean particle size of about 0.001 to about 0.750 mm, about 0.005 to about 0.150 mm, about 0.250 to about 0.750 mm, 0.250 to about 0.500, 0.015 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. The mean particle size may be bimodal to include any combination of mean particle sizes as previously described. In some embodiments, said granules may exhibit a mean granule size of about 0.050 mm to about 5 mm, about 0.100 to about 1.500 mm, about 0.125 to 1.000 mm, 0.125 to 0.500 mm, about 0.125 to 0.250 mm, about 0.250 to 0.750 mm, about 0.250 to 0.500 mm, about 0.500 to 1.00 mm, about 0.500 to 0.750 mm. The mean granule size may be multi-modal to include any combination of mean granule sizes as previously described. In some embodiments, varying sizes of said powders or granules may be used in the adhesive composition.

In some embodiments, certain additives are provided as fibers. In some embodiments, the fibers exhibit a mean fiber diameter of about 0.010 mm to about 2 mm, about 0.010 mm to about 0.50 mm, or about 0.025 mm to about 0.075 mm. These fibers may exhibit a mean fiber length of about 0.025 mm to about 5.0 mm, about 0.50 mm to 10 mm, or about 1.00 mm to about 3.50 mm. In some embodiments, the preferable fiber length is 2.0 mm. The mean fiber diameter or length may be multi-modal to include any combination of mean fiber diameter or length as previously described. In some embodiments, size can be defined by the aspect ratio, wherein the aspect ratio is from 2:1 to 100:1, or more preferably from 20:1 to 70:1.

In some embodiments, one or more additives supplied as granules or fibers are porous. In some embodiments, certain additives supplied as granules or fibers are non-porous. The porous additive may be interconnected or closed. The size of the pores in the porous additive may range in size from the nanometer range (e.g., about 10 nm to about 1000 nm) to micrometer range (e.g., about 10 μm to about 1000 μm) to the millimeter range (e.g., about 1 mm to about10 mm). The total porosity of the additive may range from about 5% porosity to about 95% porosity. In some embodiments, the porosity of the additive is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% porosity.

In some embodiments, the additive is poly-lactic-glycolic acid (PLGA). In some embodiments, PLGA is added as a component to the composition, where the PLGA increases the strength of the adhesive composition. In some embodiments, the addition of PLGA improves the composition's resistance to impact and fatigue stress and maximizes the integrity of the structural scaffold to support early repetitive load bearing.

In some embodiments, the chosen additive is added to the composition up to 30 w/w % based on the total weight of the composition to increase the intrinsic strength of the material. However, as additive levels increase, adhesive properties may decrease and therefore a balance between intrinsic strength and material adhesive properties is required.

In an embodiment, the dry components and the aqueous medium are combined through the delivery system via the method discussed below to create the adhesive composition. Upon mixing, the adhesive composition described herein may have a tacky state, which may be retained for a number of days (e.g., up to 7 days, up to 3 days, up to 1 day), up to hours (e.g., up to 12 hours, up to 4 hours, up to 1 hour), up to minutes (e.g., up to 30 minutes, up to 12 minutes, up to about 4 minutes, up to about 2 minutes, up to about 1 minute), or seconds (e.g., up to 30 seconds, up to 5 seconds, up to 2 seconds). In some embodiments, the adhesive composition develops a putty-like state after the tacky state. In some embodiments, a cement-like state follows the putty state. The combined time of the tacky state and the putty state is referred to herein as working time. In some embodiments, the adhesive composition may have a working time of up to at least 3 minutes, up to at least 5 minutes, up to at least 8 minutes, up to at least 12 minutes, or up to at least 15 minutes from initial mixing, after which time the composition will have sufficiently begun hardening.

The term “tack strength,” as used herein, refers to the strength of the adhesive composition in a tacky state. In some embodiments, the tack strength of the composition is about 10 kPa to about 250 kPa. The adhesive strength of the composition in the cement state is about 100 kPa to about 12,000 kPa. In some embodiments, the adhesive composition may begin to harden within about 8 minutes, e.g., within about 5 minutes, within about 3 minutes, or within about 15 minutes, after mixing with the aqueous medium near room or body temperature. In some embodiments, the adhesive composition is formulated to begin self-setting, e.g., harden, within a specific amount of time. In some embodiments, the adhesive composition is flowable through a cannula tip on the order of minutes, e.g., about 3 minutes, before self-setting begins.

The present disclosure further provides weight ratios of each component of the composition. In some embodiments, the multivalent metal salt (e.g., tetracalcium phosphate) is present in an amount of approximately 60% w/w volume of the dry components of the composition; the compound (e.g., phosphoserine) is present in an amount of approximately 35% w/w volume of the dry components of the composition; the contrast agent (e.g., BaSO₄) is present in an amount of approximately 2% w/w volume of the dry components of the composition. In certain embodiments, one or more optional additives, e.g., PLGA microfibers, are present in an amount of approximately 3% w/w volume of the dry components of the adhesive composition. In some embodiments, an optional additive can be present in an amount up to 30% w/w volume of the whole adhesive composition.

### Methods of Use

The present disclosure provides methods of use of the delivery system , including methods of visualization described herein for delivering the adhesive composition., therapeutic, bone regenerative composition, and/or bioresorbable composition. In some embodiments, the method of use comprises the steps of one or more of: (i) aligning or reducing a fracture; (ii) transferring one or more components of the adhesive composition from a first vessel to a chamber of a second vessel; (iii) mixing the components of the adhesive composition; (iv) transferring the adhesive composition back into the first vessel; and (v) delivering, e.g., percutaneously delivering, the adhesive composition to the wound site, e.g., bone fracture. In some embodiments, the method includes (i) or (ii). In some embodiments, the method includes (i) or (iii). In some embodiments, the method includes (ii) or (iii). In some embodiments, the method includes (ii), (iii) or (iv). In some embodiments, the method includes (i), (ii), (iii) or (iv). In some embodiments, the method includes (i), (ii), (iii), (iv), or (v). In some embodiments, the method includes (i), (ii), (iii), (iv), and (v). In some embodiments, the method further comprises monitoring the delivery of the adhesive composition to the wound site via an imaging method, such as fluoroscopy, mechanical or computed tomography, or ultrasound imaging.

The present disclosure provides a method of preparation and delivery of an adhesive composition, e.g., therapeutic, bone regenerative and/or bioresorbable composition, within an exemplary delivery system. In some embodiments, the components of the delivery system are packaged in foil packing along with a plurality of cannula tips as part of a kit to be gamma irradiated for sterilization. To prepare the adhesive composition, the first vessel may be attached to the second vessel. In some embodiments, a plurality of cannula tips are provided, e.g., cannula tips for percutaneous delivery. A cannula tip may be attached to a port, e.g., an exit port, of the first vessel. In an embodiment, the method of use further comprises the step of placing the first vessel and appropriate cannula tip at the fracture site. The method of use further comprises the step of making a percutaneous cut and inserting the cannula tip at the wound site. Following cannula insertion, the adhesive composition may be controllably delivered. In some embodiments, the method of controlled delivery may be effectuated by application of a force on a thumb pad or rotational control of cannula outlets perpendicular relative to axes of the cannula. Following delivery of the adhesive composition, the cannula tip may be removed. In some embodiments, the percutaneous cut is sutured or covered, if sutures are not necessary. In some embodiments, a cast or brace is applied to support stand-alone fixation of the adhesive composition. Said cast or brace may remain applied for a short-term duration (e.g., for days, for 1 week, for 2 weeks, for 4 weeks), or for a long-term duration (e.g., for 6 weeks, for 3 months, for 6 months).

In some embodiments, the dry components and aqueous medium of the adhesive composition are mixed in the chamber of the second vessel. In some embodiments, the mixing step further comprises: attaching the second vessel to a mixing base using an adapter; activating the adapter until the components are thoroughly mixed; and detaching the second vessel from the mixing base. In some embodiments, the aqueous medium is transferred from the first vessel to the chamber of the second vessel, wherein the mixing of the components occurs in the chamber of the second vessel. In some embodiments, the aqueous medium is delivered to the chamber of the second vessel via a force against the first vessel, e.g., via a thumb pad or rotational screw. In some embodiments, the adapter is sterile. In some embodiments, the mixing base is covered in sterile drapes prior to a procedure. In some embodiments, the adapter is activated using a switch disposed on the mixing base, where activation of the adapter engages a dual function plunger. In some embodiments, activation of the adapter engages the dual function plunger to engage one or more mixing tips or blades of the dual function plunger. In some embodiments, the adapter moves the dual function plunger longitudinally through the chamber of the second vessel to permit the one or more mixing tips or blades to mix the dry components and aqueous medium to form the adhesive composition, e.g., adhesive composition, bone regenerative composition, and/or bioresorbable composition. In some embodiments, following mixing, the adhesive composition is transferred back into the first vessel. In some embodiments, the transfer is achieved via the dual function plunger. More specifically, the dual function plunger, detached from the adapter, may push or direct the adhesive composition through the port of the second vessel and into the first vessel. In some embodiments, the adhesive composition is held within the first vessel until the delivery. In some embodiments, following mixing, the switch on the mixing base is actuated to stop mixing and the mixing tips or blades retract back into the dual function plunger. In some embodiments, the adapter is detached from the dual function plunger.

In some embodiments, the method of use comprises a step of wound site preparation prior to delivery of the adhesive composition. In some embodiments, the wound site is cleaned prior to delivering the adhesive composition, e.g., via debriding. In some embodiments, a saline solution is delivered to the wound site prior to the adhesive composition via the delivery system. For example, a minimally invasive, e.g., percutaneous, cut may be made at the wound site, e.g., bone fracture site, and the wound site cleaned, e.g., by debriding the area to remove excess bone marrow or lipids from the surface of the bone and irrigating the area with a disinfectant.

The cannula tip may be placed into the percutaneous cut and the adhesive composition controllably delivered from the first vessel via a force applied to a rotational screw or a plunger. In an embodiment, delivery of the adhesive composition is monitored via imaging of a second agent (e.g., BaSO₄) present in the adhesive composition. This monitoring may be done via 2-plane fluoroscopic imaging. Once delivery is complete the cannula may be removed and the percutaneous cut bandaged or covered. In some embodiments, the surgeon may set the distal radius in a brace to support stand-alone fixation of the adhesive composition.

The present disclosure further provides for a method of use comprising image guided delivery of an adhesive composition, e.g., adhesive composition, bone regenerative composition, and/or bioresorbable composition. In some embodiments, delivery of the adhesive composition is monitored via imaging of a secondary agent (e.g., a contrast agent) in the adhesive composition. In some embodiments, the contrast agent is BaSO₄. In some embodiments, a 2-plane fluoroscopy feedback loop is used to monitor delivery of the adhesive composition. In some embodiments, computer guiding software is used to guide the application of the adhesive composition. In some embodiments, the computer guiding software provides an alert when the target location for delivery of the adhesive composition has been reached. In some embodiments, a virtual plan for the procedure is established, allowing the computer guiding software to control the timing, direction, volume, or rate of the application of the composition directly. In some embodiments, the positioning of the device is responsive to image data acquired. In some embodiments, control of the delivery of the adhesive composition is based on visualization of the wound site. In some embodiments, the delivery of the adhesive composition is responsive to the image data acquired. In some embodiments, acquiring image data of the site comprises acquiring a first set of image data of the wound site prior to delivering the adhesive composition to the bone site and acquiring a second set of image data of the wound site after delivering the adhesive composition to the bone site. In some embodiments, acquiring the image data of the site further comprises acquiring a third set of image data of the wound site during delivering of the adhesive composition to the wound site (e.g., substantially simultaneously). In some embodiments, acquiring image data of the wound site comprises acquiring image data during delivery of the adhesive composition to the bone site (e.g., substantially simultaneously). In some embodiments, monitoring the delivery of the adhesive composition occurs substantially simultaneously with the delivery of the adhesive composition. In some embodiments, real time image data acquisition to control delivery of an adhesive composition comprises acquiring images on the order of seconds during image data acquisition. For example, images may be acquired every 2 seconds, every 3 seconds, or every 4 seconds, etc.

The present disclosure further provides a method of use of an adhesive composition, e.g., adhesive composition, bone regenerative composition, and/or bioresorbable composition, and a delivery system for treatment of a wound site, e.g., bone fracture site, with one or more additional hardware fixation devices. In some embodiments, the method of preparing the adhesive composition is as described herein. In some embodiments, a hardware fixation device and the adhesive composition is delivered to the wound site in a series of staged procedures. In some embodiments, in a first procedure, an incision is made at a fracture site and the fracture site may be cleaned, e.g., debriding the area to remove excess bone marrow or lipids from the surface of the bone and irrigating the area with disinfectant. A hardware support device, e.g., a screw, may be attached to the fracture site, to aid in fixation of the fracture. In some embodiments, the incision may be closed and allowed to heal on the order of days, e.g., about 1 day to about 3 days. In some embodiments, a second procedure comprises making a percutaneous cut at the fracture site. In some embodiments, a cannula tip is placed into the percutaneous cut an adhesive composition delivered from a first syringe to the percutaneous site via an applied force, e.g., by a thumb pad or rotational screw, applied to the first syringe. In some embodiments, the delivery of the adhesive composition is monitored using a suitable imaging device, e.g., fluoroscopic imaging of a contrast agent, BaSO₄, in the adhesive composition. In specific embodiments, the monitoring may be performed by 2-plane fluoroscopic imaging. In some embodiments, following delivery, the cannula tip may be removed and the percutaneous cut covered or bandaged.

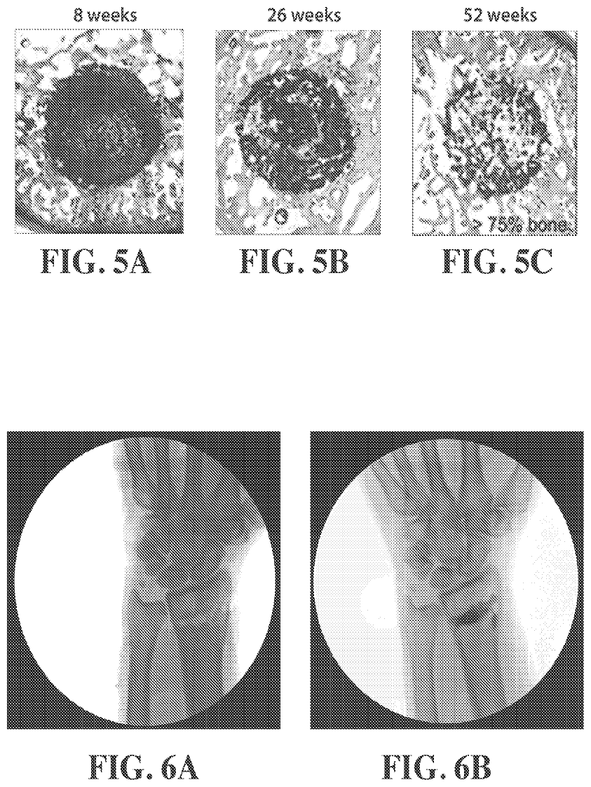

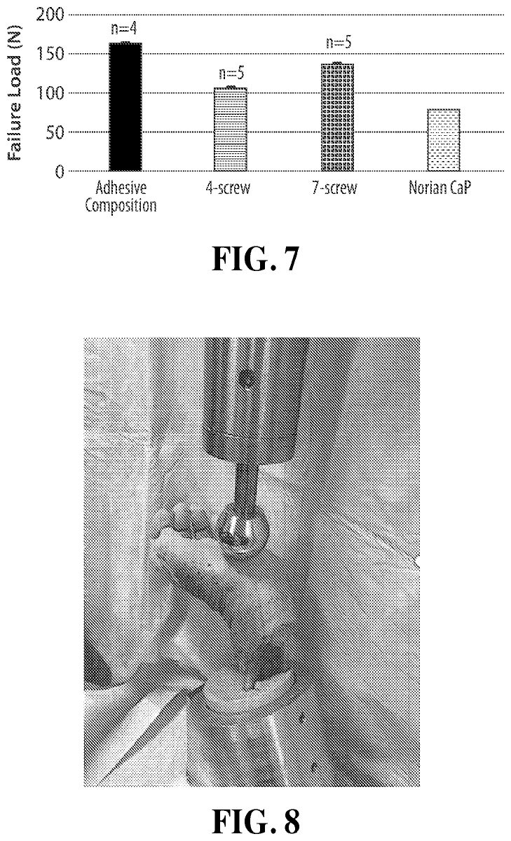

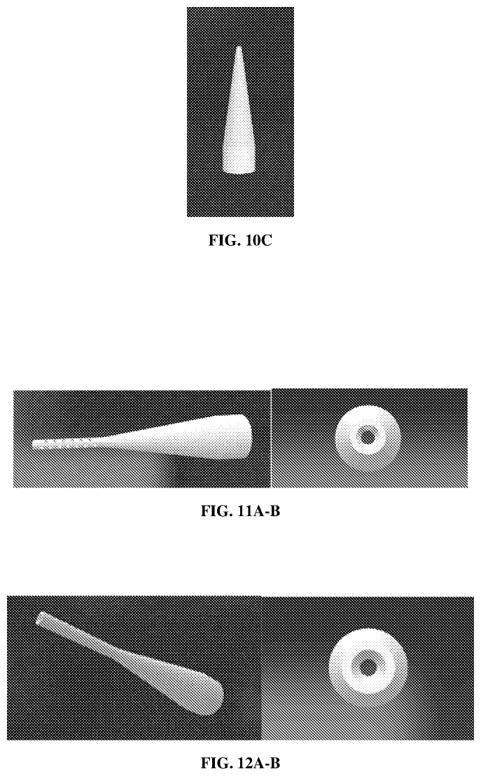

FIGS. 5A-5C illustrates three histological slides showing the material resorption of a adhesive composition, e.g., therapeutic composition, bone regenerative composition and/or bioresorbable composition as described herein, and bone replacement, in a rabbit model over a 52-week period. FIG. 5A illustrates the rabbit model at 8 weeks, FIG. 5B illustrates the rabbit model at 26 weeks, and FIG. 5C illustrates the rabbit model at 52 weeks. FIGS. 5A-5C shows the adhesive composition was well-tolerated by the rabbit body, readily replaced by natural bone, and did not impede new bone growth. FIGS. 6A-6B illustrate fluoroscopic images of a distal radius fracture before and after percutaneous delivery of a adhesive composition. FIG. 6A depicts the distal radius fracture before delivery of a adhesive composition and FIG. 6B depicts the distal radius fracture after delivery of a adhesive composition. FIG. 7 compares the compressive failure load of an exemplary adhesive composition, e.g., an adhesive composition, bone regenerative composition, and/or bioresorbable composition, to the failure load of 4-screw fixation methods, 7-screw fixation methods, and a commercially available calcium phosphate bone cement, NORIAN®. The “n=” number displayed in the graph is for the number of samples tested. FIG. 7 shows the exemplary adhesive composition has an average compressive failure load of above 150 N; 4-screw mechanical fixation has an average failure load of 100 N; 7-screw mechanical fixation has an average failure load of less than 150 N; and NORIAN® CaP bone cement has an average failure load of less than 100 N. Compressive failure is the collapse or buckling of the fracture zone resulting from compression along the fracture line. Having a higher average compressive failure load generally indicates that the fracture zone is stronger and can withstand greater compression than a fracture zone with a lower compressive failure load. FIG. 8 depicts the compressive failure load testing of a distal radius fracture site in a human cadaveric wrist. FIGS. 9A-9D depict an exemplary cannula tip connectable to the delivery system as described herein having four holes spaced evenly about the cannula tip. FIGS. 9A-9B depict views of the four holes of the cannula from a head-on view. FIGS. 9C-9D depict views of the four holes of the cannula tip from a longitudinal angle. FIGS. 10A-10C depict a cannula tip connectable to a delivery system as described herein having four large holes with four small holes spaced around the larger holes. FIGS. 10A-B depict views of the eight holes of the cannula tip from a head-on view and FIG. 10C depicts a view of the cannula tip from a longitudinal angle. FIGS. 11A-11B depict an exemplary cannula tip connectable to the delivery system as described herein, wherein the cannula tip comprises holes laterally arranged across the length of the cannula tip. FIGS. 12A-12B depict a cannula tip without holes connectable to the delivery system as described herein.

### ENUMERATED EMBODIMENTS

1. A method of image-guided delivery of a composition (e.g., an adhesive composition or a therapeutic composition) to a site (e.g., a bone site, e.g., a bone fracture site), the method comprising:
i) acquiring image data (e.g., an image) of the site (e.g., a bone site, e.g., a bone fracture site);
ii) positioning a device (e.g., a syringe, trocar, or cannula) to deliver the composition (e.g., adhesive composition or therapeutic composition) to the site (e.g., the bone site, e.g., the bone fracture site); and
iii) delivering the composition (e.g., an adhesive composition or a therapeutic composition) to the site (e.g., the bone site, e.g., a bone fracture site) via the device (e.g., the syringe, trocar, or cannula).
   2. The method of embodiment 1, wherein the composition is an adhesive composition.
   3. The method of any one of embodiments 1-2, wherein the composition is a therapeutic composition.
   4. The method of any of the preceding embodiments, wherein the site is a bone site.
   5. The method of embodiment 4, wherein the bone site is a bone fracture site.
   6. The method of any one of embodiments 4-5, wherein the bone site comprises a long bone, a short bone, a flat bone, or an irregular bone.
   7. The method of embodiment 6, wherein the bone site comprises a long bone.
   8. The method of embodiment 7, wherein the long bone is a humerus.
   9. The method of embodiment 7, wherein the long bone is a radius.
   10. The method of embodiment 7, wherein the long bone is an ulna.
   11. The method of embodiment 7, wherein the long bone is a tibia.
   12. The method of embodiment 7, wherein the long bone is a fibula.
   13. The method of embodiment 6, wherein the bone site comprises a short bone.
   14. The method of embodiment 7, wherein the short bone is a metacarpal.
   15. The method of embodiment 7, wherein the short bone is a metatarsal.
   16. The method of embodiment 6, wherein the bone site comprises a flat bone.
   17. The method of embodiment 16, wherein the flat bone is a scapula.
   18. The method of embodiment 16, wherein the flat bone is a rib.
   19. The method of embodiment 16, wherein the flat bone is a sternum.
   20. The method of embodiment 6, wherein the bone site comprises an irregular bone.
   21. The method of embodiment 20, wherein the irregular bone is a vertebral column.
   22. The method of embodiment 20, wherein the flat bone is a patella.
   23. The method of any one of the preceding embodiments, wherein the device is selected from a syringe, trocar, and cannula.
   24. The method of embodiment 23, wherein the device is a syringe.
   25. The method of embodiment 23, wherein the device is a trocar.
   26. The method of embodiment 23, wherein the device is a cannula.
   27 The method of any one of the preceding embodiments, wherein i) is performed prior to ii).
   28. The method of any one of the preceding embodiments, wherein i) is performed prior to iii).
   29. The method of any one of the preceding embodiments, wherein i) is performed after ii) but before iii).
   30. The method of any one of the preceding embodiments, wherein i) is performed after ii) and iii).
   31. The method of any one of the preceding embodiments, wherein the composition comprises a multivalent metal salt, an organic compound, and an aqueous medium.
   32. The method of embodiment 31, wherein the multivalent metal salt comprises one or more of calcium phosphates (e.g., hydroxyapatite, octacalcium phosphate, tetracalcium phosphate, tricalcium phosphate), calcium nitrate, calcium citrate, calcium carbonate, magnesium phosphates, sodium silicates, lithium phosphates, titanium phosphates, strontium phosphates, barium phosphates, zinc phosphates, calcium oxide, magnesium oxide, and combinations thereof.
   33. The method of embodiment 32, wherein the multivalent metal salt comprises tetracalcium phosphate.
   34. The method of embodiment 32 or 33, wherein the multivalent metal salt comprises tricalcium phosphate.
   35. The method of embodiment 32, wherein the multivalent metal salt comprises hydroxyapatite.
   36. The method of embodiment 32, wherein the multivalent metal salt comprises octacalcium phosphate.
   37. The method of embodiment 32, wherein the multivalent metal salt comprises calcium nitrate.
   38. The method of embodiment 32, wherein the multivalent metal salt comprises calcium citrate.
   39. The method of embodiment 32, wherein the multivalent metal salt comprises calcium carbonate.
   40. The method of embodiment 32, wherein the multivalent metal salt comprises magnesium phosphates.
   41. The method of embodiment 32, wherein the multivalent metal salt comprises sodium silicates.
   42. The method of embodiment 32, wherein the multivalent metal salt comprises lithium phosphates.
   43. The method of embodiment 32, wherein the multivalent metal salt comprises titanium phosphates.
   44. The method of embodiment 32, wherein the multivalent metal salt comprises strontium phosphates.
   45. The method of embodiment 32, wherein the multivalent metal salt comprises barium phosphates.
   46. The method of embodiment 32, wherein the multivalent metal salt comprises zinc phosphates.
   47. The method of embodiment 32, wherein the multivalent metal salt comprises calcium oxide.
   48. The method of embodiment 32, wherein the multivalent metal salt comprises magnesium oxide.
   49. The method of any one of embodiments 31-48, wherein the organic compound is a compound of any one of Formulas (I), (II), (III), (IV), (V), or (VI).
   50 The method of embodiment 49, wherein the organic compound is a compound of Formula (I):

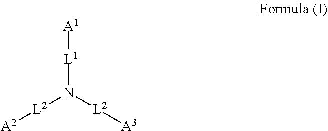

or a salt thereof, wherein:
each of A¹, A², and A³ is independently selected from an acidic group; and
each of L¹, L², and L³ is independently a bond, alkylene, or heteroalkylene.

51. The method of embodiment 34, wherein the organic compound is a compound of Formula (II):

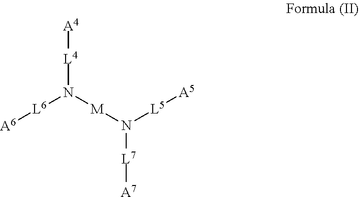

or a salt thereof, wherein:
each of A⁴, A⁵, and A⁶,is independently selected from an acidic group;
A⁷ is selected from an acidic group, a hydrogen atom, an alkyl, an aryl, a hydroxy group, a thio group, and an amino group;
each of L⁴, L⁵, L⁶, and L⁷ is independently a bond, alkylene, or heteroalkylene; and
M is alkylene or heteroalkylene.
   52. The method of embodiment 49, wherein the organic compound is a compound of Formula (III):

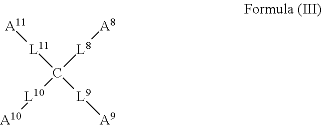

or a salt thereof, wherein:
each of A⁸ and A⁹ is independently selected from an acidic group;
each of A¹⁰ and A¹¹ is independently selected from an acidic group, a hydrogen atom, an alkyl, aryl, a hydroxy group, a thio group, and an amino group;
each of L⁸, L⁹, L¹⁰, and L¹¹ is independently a bond, alkylene, or heteroalkylene.
   53. The method of embodiment 49, wherein the organic compound is a compound of Formula (IV):

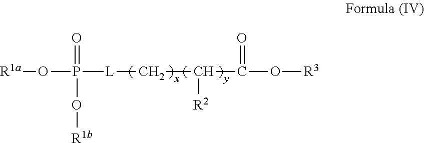

or a salt thereof, wherein:
L is O, S, NH, or CH₂;
each of R^{1a} and R^{1b} is independently H, an optionally substituted alkyl, or an optionally substituted aryl;
R² is H, NR^{4a}R^{4b}, C(O)R⁵, or C(O)OR⁵;
R³ is H, an optionally substituted alkyl, or an optionally substituted aryl;
each of R^{4a} and R^{4b} is independently H, C(O)R⁶, or an optionally substituted alkyl;
R⁵ is H, an optionally substituted alkyl, or an optionally substituted aryl;
R⁶ is an optionally substituted alkyl or an optionally substituted aryl; and
each of x and y is independently 0, 1, 2, or 3.
   54. The method of embodiment 49, wherein the organic compound is a compound of Formula (V):

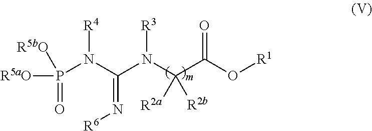

or a salt thereof, wherein:
R¹ is H, optionally substituted alkyl, optionally substituted alkenyl, optionally substituted alkynyl, optionally substituted aryl, or optionally substituted heteroaryl;
each of R^{2a} and R^{2b} is independently H, optionally substituted alkyl, hydroxy, alkoxy, or halo;
each of R³ and R⁴ is independently H or optionally substituted alkyl;
each of R^{5a} and R^{5b} is independently H, optionally substituted alkyl, optionally substituted alkenyl, optionally substituted alkynyl, optionally substituted aryl, or optionally substituted heteroaryl;
R⁶ is H or optionally substituted alkyl; and
m is 1, 2, 3, 4, or 5.
   55. The method of embodiment 49, wherein the compound is a compound of Formula (VI):

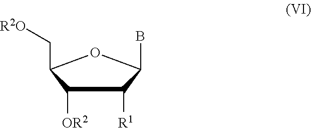

or a salt thereof, wherein:
B is a nucleobase;
R¹ is H, OR⁴, or halo;
R² is H, optionally substituted alkyl, optionally substituted alkenyl, optionally substituted alkynyl, optionally substituted aryl, optionally substituted heteroaryl, optionally substituted cycloalkyl, or optionally substituted heterocyclyl;
R³ is H, optionally substituted alkyl, or a phosphate moiety (e.g., monophosphate or diphosphate); and
R⁴ is H, optionally substituted alkyl, optionally substituted alkenyl, optionally substituted alkynyl, optionally substituted aryl, optionally substituted heteroaryl, optionally substituted cycloalkyl, or optionally substituted heterocyclyl.
   56. The method of any one of embodiments 49-55, wherein the organic compound is phosphoserine, calcium citrate, or malonic acid (e.g., phosphoserine).
   57. The method of any one the preceding embodiments, wherein the adhesive composition has a viscosity between 100 cP and 10,000 cP.
   58. The method of any one the preceding embodiments, wherein the adhesive composition further comprises an agent (e.g., a contrast agent or radioisotope).
   59. The method of embodiment 58, wherein the agent is a contrast agent.
   60. The method of embodiment 59, wherein the contrast agent comprises barium.
   61. The method of embodiment 59, wherein the contrast agent comprises iodine.
   62. The method of embodiment 59, wherein the contrast agent comprises bismuth.
   63. The method of embodiment 59, wherein the contrast agent comprises gadolinium.
   64 The method of embodiment 59, wherein the contrast agent comprises CO₂.
   65. The method of embodiment 59, wherein the contrast agent comprises hydrogen.
   66 The method of embodiment 59, wherein the contrast agent comprises air.
   67. The method of any one of embodiments 58-66, wherein the agent is a radioisotope.
   68. The method of any one of embodiments 58-67, wherein an amount of the agent in the composition is less than about 20%, 15%, 10%, 9%, 8%, 7%, 6%, 5%, 4%, 3%, 2%, 1%, 0.5%, 0.1%, 0.05%, or 0.01% (w/w or w/v) of the total adhesive composition.
   69. The method of any one of the preceding embodiments, the composition has a tacky state for up to 12 minutes.
   70. The method of embodiment 69, the tacky state has a tack stress of about 10 kPa to about 250 kPa.
   71. The method of any one of the preceding embodiments, wherein the composition has an adhesive strength upon curing of about 100 kPa to about 12,000 kPa.
   72. The method of any one of the preceding embodiments, wherein the composition has a viscosity of about 100 cP to about 10,000 cP when in a fluid state.
   73. The method of any one of the preceding embodiments, wherein the composition has a viscosity of about 10,000 cP to about 250,000 cP when in a semi-solid or tack state.
   74 The method of any one of embodiments 72-73, wherein the viscosity of the composition is such that the composition does not substantially extravasate into surrounding tissues during delivery.
   75. The method of any one of embodiments 31-74, wherein the amount of the multivalent metal compound is about 10% to about 90% weight by weight (w/w) of the composition.
   76. The method of any one of the preceding embodiments, wherein the image data comprises one or more images.
   77 A method of joining two or more (e.g., three, four, five, six, seven, eight, nine, or ten) bone fragments together at a site (e.g., a trauma site or surgical site), the method comprising:
i) acquiring image data (e.g., an image) of the site (e.g., a trauma site or surgical site);
ii) positioning a device (e.g., syringe or trocar or cannula) constructed and arranged to deliver a composition (e.g., an adhesive composition or a therapeutic composition) to the site (e.g., a trauma site or surgical site); and
iii) delivering the composition (e.g., an adhesive composition or a therapeutic composition) to the site (e.g., a trauma site or surgical site) via the device (e.g., syringe or trocar or cannula).
   78. The method of embodiment 77, wherein the composition is an adhesive composition.
   79. The method of any one of embodiments 77-78, wherein the composition is a therapeutic composition.
   80. The method of any one of embodiments 79, wherein the site is a trauma site.
   81. The method of embodiment 79, wherein the site is a surgical site.
   82. The method of any one of embodiments 77-81, wherein the device is selected from a syringe, trocar, and cannula.
   83. The method of embodiment 82, wherein the device is a syringe.
   84. The method of embodiment 82, wherein the device is a trocar.
   85. The method of embodiment 82, wherein the device is a cannula.
   86. The method of any one of embodiments 77-85, wherein i) is performed prior to ii).
   87. The method of any one of embodiments 77-86, wherein i) is performed prior to iii).
   88. The method of any one of embodiments 77-87, wherein i) is performed after ii) but before iii).
   89. The method of any one of embodiments 77-88, wherein i) is performed after ii) and iii).
   90. The method of any one of embodiments 77-89, wherein the composition comprises a multivalent metal salt, an organic compound, and an aqueous medium.
   91. The method of embodiment 90, wherein the multivalent metal salt comprises one or more of calcium phosphates (e.g., hydroxyapatite, octacalcium phosphate, tetracalcium phosphate, tricalcium phosphate), calcium nitrate, calcium citrate, calcium carbonate, magnesium phosphates, sodium silicates, lithium phosphates, titanium phosphates, strontium phosphates, barium phosphates, zinc phosphates, calcium oxide, magnesium oxide, and combinations thereof.
   92. The method of embodiment 91, wherein the multivalent metal salt comprises tetracalcium phosphate.
   93. The method of embodiment 91, wherein the multivalent metal salt comprises tricalcium phosphate.
   94. The method of embodiment 91, wherein the multivalent metal salt comprises hydroxyapatite.
   95. The method of embodiment 91, wherein the multivalent metal salt comprises octacalcium phosphate.
   96 The method of embodiment 91, wherein the multivalent metal salt comprises calcium nitrate.
   97. The method of embodiment 91, wherein the multivalent metal salt comprises calcium citrate.
   98. The method of embodiment 91, wherein the multivalent metal salt comprises calcium carbonate.
   99. The method of embodiment 91, wherein the multivalent metal salt comprises magnesium phosphates.
   100. The method of embodiment 91, wherein the multivalent metal salt comprises sodium silicates.
   101. The method of embodiment 91, wherein the multivalent metal salt comprises lithium phosphates.
   102. The method of embodiment 91, wherein the multivalent metal salt comprises titanium phosphates.
   103. The method of embodiment 91, wherein the multivalent metal salt comprises strontium phosphates.
   104. The method of embodiment 91, wherein the multivalent metal salt comprises barium phosphates.
   105. The method of embodiment 91, wherein the multivalent metal salt comprises zinc phosphates.
   106. The method of embodiment 91, wherein the multivalent metal salt comprises calcium oxide.
   107. The method of embodiment 91, wherein the multivalent metal salt comprises magnesium oxide.
   108. The method of any one of embodiments 90-107, wherein the organic compound is a compound of any one of Formulas (I), (II), (III), (IV), (V), or (VI).
   109. The method of embodiment 108, wherein the organic compound is a compound of Formula (I):

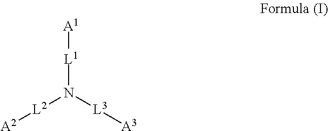

or a salt thereof, wherein:
each of A¹, A², and A³ is independently selected from an acidic group; and
each of L¹, L², and L³ is independently a bond, alkylene, or heteroalkylene.
   110. The method of embodiment 108, wherein the organic compound is a compound of Formula (II):

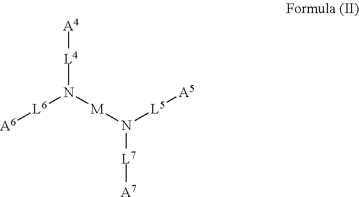

or a salt thereof, wherein:
each of A⁴, A⁵, and A⁶, is independently selected from an acidic group;
A⁷ is selected from an acidic group, a hydrogen atom, an alkyl, an aryl, a hydroxy group, a thio group, and an amino group;
each of L⁴, L⁵, L⁶, and L⁷ is independently a bond, alkylene, or heteroalkylene; and
M is alkylene or heteroalkylene.
   111. The method of embodiment 108, wherein the organic compound is a compound of Formula (III):

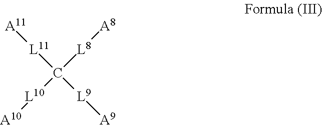

or a salt thereof, wherein:
each of A⁸ and A⁹ is independently selected from an acidic group; each of A¹⁰ and A¹¹ is independently selected from an acidic group, a hydrogen atom, an alkyl, aryl, a hydroxy group, a thio group, and an amino group; each of L⁸, L⁹, L¹⁰ and L¹¹ is independently a bond, alkylene, or heteroalkylene.
   112. The method of embodiment 108, wherein the organic compound is a compound of Formula (IV):

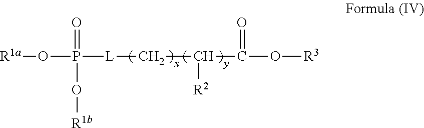

or a salt thereof, wherein:
L is O, S, NH, or CH₂;
each of R^{1a} and R^{1b} is independently H, an optionally substituted alkyl, or an optionally substituted aryl;
R² is H, NR^{4a}R^{4b}, C(O)R⁵, or C(O)ORR⁵;
R³ is H, an optionally substituted alkyl, or an optionally substituted aryl; each of R^{4a} and R^{4b} is independently H, C(O)R⁶, or an optionally substituted alkyl;
R⁵ is H, an optionally substituted alkyl, or an optionally substituted aryl;
R⁶ is an optionally substituted alkyl or an optionally substituted aryl; and
each of x and y is independently 0, 1, 2, or 3.
   113. The method of embodiment 108, wherein the organic compound is a compound of Formula (V):

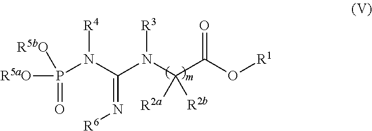

or a salt thereof, wherein:
R¹ is H, optionally substituted alkyl, optionally substituted alkenyl, optionally substituted alkynyl, optionally substituted aryl, or optionally substituted heteroaryl;
each of R^{2a} and R^{2b} is independently H, optionally substituted alkyl, hydroxy, alkoxy, or halo;
each of R³ and R⁴ is independently H or optionally substituted alkyl;
each of R^{5a} and R^{5b} is independently H, optionally substituted alkyl, optionally substituted alkenyl, optionally substituted alkynyl, optionally substituted aryl, or optionally substituted heteroaryl;
R⁶ is H or optionally substituted alkyl; and
m is 1, 2, 3, 4, or 5.
   114. The method of embodiment 108, wherein the compound is a compound of Formula (VI):

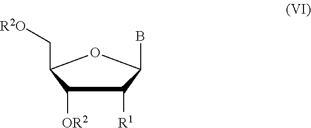

or a salt thereof, wherein:
B is a nucleobase;
R¹ is H, OR⁴, or halo;
R² is H, optionally substituted alkyl, optionally substituted alkenyl, optionally substituted alkynyl, optionally substituted aryl, optionally substituted heteroaryl, optionally substituted cycloalkyl, or optionally substituted heterocyclyl;
R³ is H, optionally substituted alkyl, or a phosphate moiety (e.g., monophosphate or diphosphate); and
R⁴ is H, optionally substituted alkyl, optionally substituted alkenyl, optionally substituted alkynyl, optionally substituted aryl, optionally substituted heteroaryl, optionally substituted cycloalkyl, or optionally substituted heterocyclyl.
   115. The method of any one of embodiments 108-114, wherein the organic compound is calcium citrate, malonic acid, or phosphoserine (e.g., phosphoserine).
   116. The method of any one the embodiments 77-115, wherein the adhesive composition has a viscosity between 100 cP and 10,000 cP.
   117. The method of any one the embodiments 77-116, wherein the adhesive composition further comprises an agent (e.g., a contrast agent or radioisotope).
   118. The method of embodiment 117, wherein the agent is a contrast agent.
   119. The method of embodiment 118, wherein the contrast agent comprises barium.
   120. The method of embodiment 118, wherein the contrast agent comprises iodine.
   121. The method of embodiment 118, wherein the contrast agent comprises bismuth.
   122. The method of embodiment 118, wherein the contrast agent comprises gadolinium.
   123. The method of embodiment 118, wherein the contrast agent comprises CO₂.
   124. The method of embodiment 118, wherein the contrast agent comprises hydrogen.
   125. The method of embodiment 118, wherein the contrast agent comprises air.
   126. The method of any one of embodiments 117-125, wherein the agent is a radioisotope.
   127. The method of any one of embodiments 117-126, wherein an amount of the agent in the composition is less than about 20%, 15%, 10%, 9%, 8%, 7%, 6%, 5%, 4%, 3%, 2%, 1%, 0.5%, 0.1%, 0.05%, or 0.01% (w/w or w/v) of the total adhesive composition.
   128. The method of any one of embodiments 77-127, the composition has a tacky state for up to 12 minutes.
   129. The method of embodiment 128, the tacky state has a tack stress of about 10 kPa to about 250 kPa.
   130. The method of any one of embodiments 77-129, wherein the composition has an adhesive strength upon curing of about 100 kPa to about 12,000 kPa.
   131. The method of any one of embodiments 77-130, wherein the composition has a viscosity of about 100 cP to about 10,000 cP when in a fluid state.
   132. The method of any one of embodiments 77-131, wherein the composition has a viscosity of about 10,000 cP to about 250,000 cP when in a semi-solid or tack state.
   133. The method of any one of embodiments 131-132, wherein the viscosity of the composition is such that the composition does not substantially extravasate into surrounding tissues during delivery.
   134. The method of any one of embodiments 90-133, wherein the amount of the multivalent metal compound is about 10% to about 90 weight by weight (w/w) of the composition.
   135. The method of any one of the preceding embodiments, wherein the image data is one or more images.
   136. The method of any one of the preceding embodiments, wherein the positioning of the delivery device is responsive to the image data acquired.
   137. The method of any one of the preceding embodiments, wherein the delivering of the composition is responsive to the image data acquired.
   138. The method of any one of the preceding embodiments, wherein delivering the composition further comprises controlling the delivery of the composition to the wound site.
   139. The method of embodiment 138, wherein controlling the delivery of a composition comprises controlling one or more of: the amount of the composition delivered, the location of delivery of the composition, the duration of delivery of the composition, or the flow rate of delivery of the composition.
   140. The method of any one of embodiments 138-139, wherein controlling the delivery of a composition comprises controlling the amount of composition delivered.
   141. The method of any one of embodiments 138-140, wherein controlling the delivery of a composition comprises controlling the location of delivery of the composition.
   142. The method of any one of embodiments 138-141, wherein controlling the delivery of a composition comprises controlling the duration of delivery of the composition.
   143. The method of any one of embodiment 138-142, wherein controlling the delivery of a composition comprises controlling the flow rate of delivery of the composition.
   144. The method of any one of the preceding embodiments, further comprising comparing the amount of the composition delivered to the site to a desired quantity of the composition to be delivered to the site.
   145. The method of any one of the preceding embodiments, wherein the acquiring of i) comprises directly acquiring the image data or indirectly acquiring the image data.
   146. The method of any one of the preceding embodiments, wherein the acquiring of i) comprises acquiring a first set of image data of the site prior to delivering the composition to the site.
   147. The method of any one of embodiments 145-146, wherein the acquiring of i) comprises acquiring a second set of image data of the wound site after delivering the composition to the site.
   148. The method of any one of embodiments 145-147, further comprising acquiring a third set of image data of the site while delivering the composition to the wound site.
   149. The method of any one of the preceding embodiments, wherein acquiring image data comprises directly acquiring the image data.
   150. The method of any one of the preceding embodiments, wherein acquiring image data comprises indirectly acquiring the image data.
   151. The method of any one of the preceding embodiments, wherein acquiring image data comprises acquiring image data via a two-dimensional imaging method.
   152. The method of any one of the preceding embodiments, wherein acquiring image data comprises acquiring image data via a three-dimensional imaging method.
   153. The method of any one of the preceding embodiments, wherein acquiring of image data comprises acquiring data related to heat.
   154. The method of any one of the preceding embodiments, wherein acquiring of image data comprises acquiring data related to the position of sound waves.
   155. The method of any one of the preceding embodiments, wherein acquiring of image data comprises acquiring data related to the position of X-rays.
   156. The method of any one of the preceding embodiments, wherein acquiring of image data comprises acquiring data related to the position of radio waves.
   157. The method of any one of the preceding embodiments, wherein acquiring of image data comprises acquiring data related to the presence of a contrast agent.
   158. The method of any one of the preceding embodiments, wherein acquiring of image data comprises fluoroscopy.
   159. The method of any one of the preceding embodiments, wherein acquiring of image data comprises mechanical tomography.
   160. The method of any one of the preceding embodiments, wherein acquiring of image data comprises computed tomography (CT).
   161. The method of any one of the preceding embodiments, wherein acquiring of image data comprises magnetic resonance imaging.
   162. The method of any one of the preceding embodiments, wherein acquiring of image data comprises X-ray
   163. The method of any one of the preceding embodiments, wherein acquiring of image data comprises ultrasound.
   164. The method of any one of the preceding embodiments, wherein acquiring of image data comprises positron emission tomography (PET).
   165. The method of any one of the preceding embodiments, wherein acquiring of image data comprises scintigraphy.
   166. The method of any one of embodiments 158-165, wherein acquiring image data comprises operating an ultrasound wand.
   167. The method of any one of embodiments 158-165, wherein acquiring image data comprises operating a fluoroscope.
   168. The method of any one of embodiments 158-165, wherein acquiring image data comprises operating mechanical tomography equipment.
   169. The method of any one of embodiments 158-165, wherein acquiring image data comprises operating an X-ray generator.
   170. The method of any one of the preceding embodiments, wherein acquiring of image data comprises use of a computer-assisted imaging system.
   171. The method of embodiment 170, wherein use of a computer-assisted imaging system comprises acquiring signal data from a wound site.
   172. The method of any one of embodiments 170-171, wherein use of a computer-assisted imaging system comprises converting the signal data to image data.
   173. The method of any one of embodiments 170-172, wherein use of a computer-assisted imaging system comprises displaying the image data to a user.
   174. The method of any one of embodiments 170-173, wherein use of a computer-assisted imaging system comprises:
i) acquiring signal data from a wound site;
ii) converting the signal data to image data; or iii) displaying the image data to a user.
   175. The method of any one of embodiments 170-174, wherein use of a computer-assisted imaging system comprises:
i) acquiring signal data from a wound site;
ii) converting the signal data to image data; and
iii) displaying the image data to a user.
   176. The method of any one of the preceding embodiments, further comprising iv) making an incision in a subject.
   177. The method of embodiment 176, wherein the making of the incision is responsive to acquiring image data.
   178. The method of any one of embodiments 176-177, wherein the delivering of the composition occurs following making the incision in the subject.
   179. The method of any one of embodiments 176-178, wherein the delivering of the composition comprises delivering the composition percutaneously via the incision.
   180. The method of any one of embodiments 176-179, wherein delivering of the composition comprises delivering the composition to the site via a lumen of the device.
   181. The method of embodiment 180, wherein the lumen is defined within a wall of the device having an outer dimension between about 5 mm to about 10 mm.
   182. The method of embodiment 181, wherein a length of the wall of the lumen is about 30 mm to about 300 mm.
   183. The method of any one of the preceding embodiments, further comprising referencing one or more fiducial features on one or more components of the device.
   184. The method of any one of the preceding embodiments, wherein the delivery system further comprises one or more cannula tips for delivering the composition to the site.
   185. The method of any one of the preceding embodiments, further comprising providing an alert when the device has reached the site.
   186. The method of any one of the preceding embodiments, wherein the device is constructed and arranged to direct a flow of the composition at an angle relative to a long axis of the device.
   187. The method of any one of the preceding embodiments, wherein the device is constructed and arranged to direct a flow of the composition concentrically and parallel to the long axis of the device.
   188. The method of any one of embodiments 184-187, wherein the one or more cannula tips comprise a detectable registration feature used to indicate the trajectory opening or path of the composition during delivery.
   189. The method of any one of the preceding embodiments, wherein the delivery system further comprises a balloon.
   190. The method of any one of the preceding embodiments, wherein the delivery system further comprises a mesh containment device.
   191. The method of any one of embodiments 189-190, wherein the balloon or mesh containment device is microporous.
   192. The method of any one of the preceding embodiments, wherein the composition is a composition that stimulates bone regeneration.
   193. The method of any one of the preceding embodiments, wherein the composition is a pain control composition.
   194. The method of any one of the preceding embodiments, wherein the composition is an antimicrobial composition.

### EXAMPLES

The function and advantages of these and other embodiments can be better understood from the following examples. These examples are intended to be illustrative in nature and are not considered to be in any way limiting the scope of the invention.

### Example 1: Exemplary Adhesive Compositions

Exemplary adhesive compositions are outlined in Table 1A, and these exemplary compositions may comprise the exemplary organic compounds recited in Table 1B. The solid components listed in Table 1A may be combined in a suitable receptacle and mixed with sterile water for up to 3 minutes to achieve the desired consistency. The resulting properties such as viscosity, working time, setting time, and cohesive and adhesive strength, would be affected by which components are selected. The viscosity of the adhesive compositions when in its fluid state may range from as low as about 100 cP to about 10,000 cP and in its semi-solid state from about 10,000 cP to about 250,000 cP. In some embodiments, the viscosity and cohesion properties of the composition may facilitate the ability to squeeze the material through a needle or cannula (e.g., a 6, 8, 10, 12, 14, 16, 18, 20, 22, or 24-gauge needle or cannula), e.g., when the viscosity is in the low range of its fluid state without causing powder separation from the liquid components of the adhesive composition when injection forces to inject or deliver the material through a cannula to the site exceed 100 N. The adhesive compositions listed in Table 1A comprise an adhesive strength upon curing of greater than 100 kPa (e.g., greater than 150 kPa, 200 kPa, 250 kPa, 300 kPa, 400 kPa, 500 kPa, 600 kPa, 750 kPa, 1000 kPa or more). The specific mean particle and or granule size for each solid component is selected to satisfy the use requirements as described in each of the embodiments. The components are supplied to the user in protective packaging and provided along with sterile components previously sterilized using techniques such as gamma irradiation.

The quantities of each of the components listed may be altered or adjusted in relation to the other components in the composition. After mixing, the compositions described may be applied to the desired site using percutaneous injection through a delivery syringe. The user monitored application of the composition to the desired site via imaging of the imaging agent in the composition, e.g., via fluoroscopy, MRI imaging, or ion imaging.

TABLE 1A
Exemplary Adhesive Compositions

| | | Compositions | | | | | | | | | | |
|---|---|---|---|---|---|---|---|---|---|---|---|---|
| Component | Units | A | B | C | D | E | F | G | H | I | J | K |
| Tetracalcium | mg | 400 | 400 | 400 | 400 | 400 | 400 | 400 | 400 | 400 | 400 | 400 |
| phosphate^{1, 2} | | | | | | | | | | | | |
| O-phosho-L-serine^{3, 4} | mg | 250 | 250 | 300 | 250 | 250 | 250 | 250 | 250 | 250 | 250 | 250 |
| Calcium carbonate⁵ | mg | — | 50 | — | — | 50 | — | 50 | — | — | 25 | — |
| Preformed porous | mg | — | — | — | — | — | 100 | — | — | 150 | 100 | 150 |
| granules⁶ | | | | | | | | | | | | |
| PLGA (10:90) fibers^{7, 8} | mg | — | — | — | — | — | — | 35 | 35 | 35 | 35 | 0 |
| Barium sulfate⁹ | mg | — | — | — | 25 | 25 | 25 | 25 | 25 | 25 | 25 | 25 |
| Water¹⁰ | μl | 130 | 135 | 140 | 140 | 150 | 170 | 160 | 175 | 175 | 180 | 175 |

¹Particle size, average 20-40 μm; however, other particle size distributions could be used per detailed description to control curing kinetics and strength
²Other multivalent metal salts, or combinations of multivalent metal salts, could be used per detailed description (e.g., α-tricalcium phosphate, hydroxyapatite, calcium oxide, and/or calcium carbonate).
³Particle size, mean 5-15 μm; however, other particle size distributions could be used per detailed description to control curing kinetic and strength
⁴Other compounds could be used per detailed description instead of O-phospho-L-serine, e.g., Table 1B
⁵Particle size, average 15-40 nm; however, other particle size distributions could be used per detailed description to control size of pores and percent porosity created during setting reaction
⁶As produced in Example 2; other porous granules could be used per detailed description, e.g., hydroxyapatite, beta-tricalcium phosphate
⁷PLGA = (Poly)lactic-glycolic-acid, comprised of 10% lactic acid and 90% glycolic acid, 35 μm diameter × 1.75 mm length
⁸Other resorbable fibers could be used per detailed description, e.g., collagen, PLGA (50:50), PLGA (90:10) and at different diameters and lengths per detailed description
⁹Other imaging agents could be used per detailed description, e.g., iodine-based
¹⁰Any aqueous based medium could be used per detailed description, e.g., blood, PBS, saline, serum, 1-5% NaOH

TABLE 1B
Exemplary Organic Compounds

| Formula | Compound Name | Structure |
|---|---|---|
| I-a | N,N-Bis(phosphonomethyl)glycine (Glyphosine) | 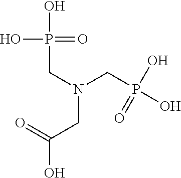 [SMILES: O=C(O)CN(CP(=O)(O)O)CP(=O)(O)O] |
| I-b | (Nitrilotris(methylene))triphosphonic acid (ATMP) | 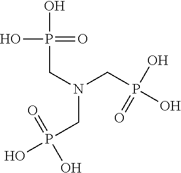 [SMILES: O=P(O)(O)CN(CP(=O)(O)O)CP(=O)(O)O] |
| II-a | Ethylenediamine tetraacetic acid (EDTA) | 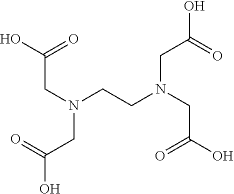 [SMILES: O=C(O)CN(CCN(CC(=O)O)CC(=O)O)CC(=O)O] |
| II-b | Egtazic acid (EGTA) | 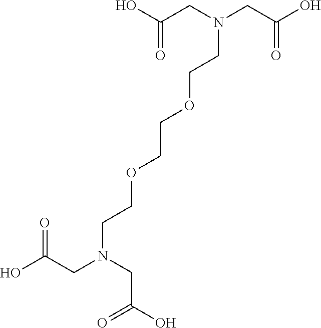 [SMILES: O=C(O)CN(CCOCCOCCN(CC(=O)O)CC(=O)O)CC(=O)O] |
| II-c | Propylenediaminetetraacetic acid (PDTA) | 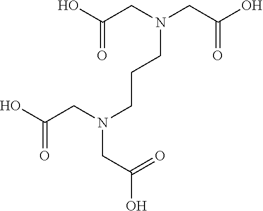 [SMILES: O=C(O)CN(CCCN(CC(=O)O)CC(=O)O)CC(=O)O] |
| III-a | Malonic Acid | 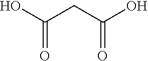 |
| III-b | Citric Acid | 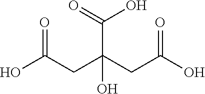 [SMILES: O=C(O)CC(O)(CC(=O)O)C(=O)O] |
| IV-a | O-Phospho-L-Serine (OPLS) | 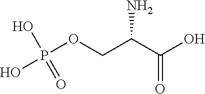 [SMILES: N[C@@H](COP(=O)(O)O)C(=O)O] |
| V-a | Phosphocreatine | 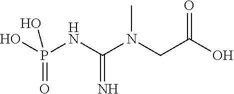 |
| VI-a | 2′-Deoxyadenosine monophosphate | 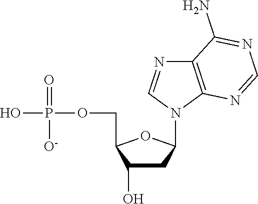 |
| VI-b | 2′-Deoxyadenosine diphosphate | 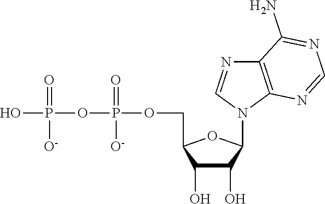 |

### Example 2: Solidified Form of Adhesive Composition in the Form of Porous Granules

Exemplary porous granules were produced by mixing a multiple of 10 times the components of Adhesive Composition B described in Example 1, Table 1A using a spatula for 20 to 30 seconds in a 25 mL silicone mixing vessel to form a homogenous adhesive slurry. The mixing vessel holding the slurry was gently tapped to allow the material to settle and form a level surface within. The slurry was cured for 10 minutes at room temperature before being transferred to a humidity chamber set to 50-65° C. for an additional 15-60 minutes. The solidified adhesive composition was removed as a porous block from the mixing bowl and broken into several wedges using an Arbor press. The wedges of material were processed through a jaw crusher (i.e., Reutsch Model # BB50) first through a 5 mm gap setting. The material was collected and re-processed again through the jaw crusher, but with a 2 mm gap setting. The collected material was next processed through a co-mill (Quattro Model #193) selected to reduce the porous granule size to the desired size range. In this example, the material was co-milled at 750 rpm through a stainless screen with mesh size of 4,750 μm and a spacer of 0.3″. The porous granules were collected again and re-processed through the co-mill in a similar fashion, but with a series of reduced mesh size stainless steel screens ranging from larger mesh (2,388 μm) run at 750 rpm to smaller mesh (610 μm) run at 2,000 rpm. The granules were collected and sieved between stainless screens of 105 μm to 500 μm. Other sieves could be used for different size ranges. The granules were packed for storage to protect the material from moisture contamination. These granules were utilized as an additive in the adhesive compositions disclosed. When these granules were used as an additive in an adhesive composition, the granules encouraged increased porosity through the composition which enhances the rate of resorption and regeneration. The user made a percutaneous cut at the desired location and delivered the adhesive composition to the site, monitoring internal delivery of the composition via imaging of the contrast agent present in the adhesive composition, i.e., via fluoroscopy. The image data acquisition consisted of images of the bone fracture site taken every 1.5 seconds while the adhesive composition was delivered to ensure accurate delivery.

### Example 3: Treatment of a Distal Radius Fracture in a Cadaver

Using the adhesive composition labeled Composition H in Table 1A in Example 1 disclosed herein, an anatomically relevant human cadaver study was conducted where a distal radius fracture was treated. Four cadaveric wrist specimens were fixated with the adhesive composition loaded with barium sulfate as an imaging agent and PLGA microfibers to increase the load strength of the fracture site. An extra articular fracture was created to simulate a Colles fracture or equivalent compression fracture in the osteoporotic cadavers. A surgeon created the necessary fracture in the cadaver bone by scoring the region of interest with a 1.6 mm diameter K wire continuously to weaken the anticipated fracture site. A large compressive force was then applied to the weakened bone until a fracture occurred in the region of interest. An x-ray of the fracture site was taken per standard clinical practice. The adhesive composition was then administered via a minimally invasive percutaneous cut via a specialized cannula tip under fluoroscopy to ensure proper placement and adherence of the adhesive composition to the fracture. FIGS. 6A-6B show x-ray images of the distal radius fracture site before and after delivery of the adhesive composition. During the studies, it was shown that the percutaneous delivery of the adhesive composition selected to adhere fractured bone fragments can reduce surgical time and complexity.

These specimens were tested under compressive loading using a servo-hydraulic load frame. The radius and ulna were exposed, and all soft tissues were removed from approximately 65 mm proximal to the wrist and the bones were potted and fixed. The proximal radius was fixed at approximately 30 degrees from the vertical, and a compressive load was applied on the proximal margin of the palm approximately against the trapezoid/capitate bone using a 35 mm spherical actuator. The compressive load was applied under displacement-controlled method at a fixed rate of 5 mm/s and along the vertical direction until failure of the fracture site. The load failure results of the four specimens fixated with the exemplary adhesive composition were compared to load failure tests of fracture sites fixated with NORIAN®, a commercially available calcium phosphate bone cement, and plate and screw volar fixation. The study in which NORIAN® was used to fixate the fracture was loaded vertically, as opposed to the others being loaded 30 degrees from vertical. The results of the NORIAN® study are best case because the load was purely a compressive load as opposed to loading at 30 degrees which multiplies the stress on the region of interest. The results are illustrated in FIG. 7, and it can be concluded that the adhesive composition provided the greatest strength for cadaveric distal radius fracture fixation compared to plates and screws and NORIAN® cement used to fill voids and augment internal hardware fixation. FIG. 9 shows that the adhesive composition has compressive load failure at 165 N, whereas 4-screw fixation has compressive load failure at 108 N, and 7-screw fixation has compressive load failure at 139 N. The adhesive composition provided greater compressive load strength and the presence of a contrast agent makes delivery of the composition easier for the surgeon.

### Example 4: Vertebral Kyphoplasty With Image Guided Delivery of an Adhesive Composition in Human Cadaver

Cadaveric studies were conducted using percutaneous delivery of adhesive composition into a vertebral compression fracture (VCF). Following the percutaneous delivery of the adhesive composition into human osteoporotic cadaveric vertebrae, the null hypothesis (p<0.05) for the following milestones will be rejected. Goals for this investigation included i) that the material extravasation of the adhesive composition was equal to or greater than vertebrae treated with a poly(methyl methacrylate) (PMMA) bone cement and ii) the mean stiffness and subsidence (height loss) of the vertebrae treated with the adhesive composition was not less than vertebrae treated with PMMA bone cement.

Intact Vertebrae Stiffness and VCF Creation: Samples used for testing were harvested from seven (7) osteoporotic cadaveric spine donors. Each sample consisted of two functional spinal units and three adjacent samples were harvested from the mid-lower thoracic spine as identified using DEXA scans for a total of 21 spine samples available for testing. Additional samples were harvested from these donors for pilot testing. Prior to testing, the height of the central vertebra was measured and averaged from four locations (anterior, posterior, transverse right, and transverse left) using digital calipers. Anteroposterior (AP) and lateral fluoroscopy radiographs of the center vertebra were taken prior to the initiation of testing. Samples were mounted within an Instron ElectroPuls E1000 testing machine. The inferior vertebra was rigidly attached to the lower crosshead of the test frame while the superior vertebra was attached to the actuator via a universal joint. A constant preload equal to 60% of the cadaver body weight was first be applied to the samples for 2 minutes. Samples underwent sub-yield point compressive loading to determine the intact stiffness of vertebrae in general accordance with the methods described in the latest revision of ASTM F20771. The vertebrae were then further compressed at a constant displacement rate of 10 mm/min until there has been a 50% decrease in the height of the center vertebrae or a yield point was observed on the load-displacement curve. The height of the center vertebra was re-measured and the change in height recorded. AP and lateral fluoroscopy radiographs were again taken, and any visible fractures sites noted.

Following fracture creation, the 21 spine samples were divided into treatment groups. Samples were distributed into the groups such that the average BMD and vertebral level was roughly equivalent between them. All non-ligamentous soft tissue was removed, and the ligaments, intervertebral disc, and all bony structures were left intact. A kyphoplasty procedure was performed on the middle segment of each sample by injecting exemplary adhesive compositions, e.g., Composition I from Table 1A in Example 1, under fluoroscopic control (Philips BV29 Fluoro C-Arm) using an inflatable bone tamp (Medtronic, Dublin, Ireland) inserted through both pedicles into the vertebrae to infiltrate the cancellous bone to restore its height. The adhesive composition was allowed to cure for 24 hours at room temperature. Some of the samples were treated with PMMA bone cement for comparison purposes.

Extravasation Assessment: Upon completion of the procedure, the incidence of extravasation was assessed using post-treatment fluoroscopy radiographs, and the degree of cement extravasation was classified into three categories—minor, moderate, and severe—based on the quantity of cement leakage. The location of any extravasation was also recorded.

Biomechanical Evaluation (Cyclic and Static Compression): Treated samples underwent cyclic fatigue testing. Samples were mounted within the Instron ElectroPuls E10000 Tension/Compression Testing Machine for uniaxial compression testing. Prior to the initiation of cyclic testing, modified uniaxial compression testing was performed to obtain the new stiffness of the treated vertebra. Samples underwent the same preload procedure as before and were then compressed at a constant displacement rate of 10 mm/min until a load equal to half of the yield load was achieved. Samples were cyclically loaded at an R ratio of 10 and a frequency of 5 Hz to a peak compressive load of 600 N. Samples were run until functional failure occurs or a maximum of 100,000 cycles was reached (i.e., “run-out”). Load and displacement data was collected digitally throughout the test. Subsidence of the vertebra, represented by the change in displacement of the test frame actuator, was recorded every 20,000 cycles. The maximum number of cycles reached, the force value at failure, and the failure mode was recorded for each sample. Height measurements and fluoroscopy radiographs were taken and any changes were noted. Following treatment and subsequent cyclic fatigue testing, uniaxial compression testing was performed to determine the stiffness of the treated, fatigued vertebrae. The same setup and procedure previously described was utilized. Height measurements and fluoroscopy radiographs were taken, and any changes noted.

### Example 5: Treatment of a Vertebral Body Defect in a Sheep Model

Using an established sheep model, an exemplary adhesive composition will be injected into a defect created within a vertebral body through a minimally invasive, e.g., percutaneous cut, transpedicular approach under fluoroscopy. The study evaluated the following: (a) compression strength confirmed using ex vivo biomechanical testing and (b) material extravasation examined through in vivo blood gas monitoring and CT imaging. This study aimed to demonstrate the rejection of the null hypothesis (p<0.05) for the following milestones. Goals for this investigation included: i) a material extravasation of the adhesive composition equal to or greater than vertebrae treated with PMMA bone cement and ii) the mean stiffness and subsidence (i.e., height loss) of the vertebrae treated with adhesive composition being not equivalent to the non-treated experimental control and not less than vertebrae treated with PMMA bone cement.

Animal Model Justification: Sheep models are widely accepted for investigating various treatments to regenerate bone and a minimally invasive transpedicular approach under fluoroscopy has been performed in multiple studies.

An in vivo study was conducted per Table 3 to obtain performance of an adhesive composition for treating bone defects using image guidance in comparison to PMMA bone cement.

TABLE 3
Pilot Sheep Study Design

| | | | Time | Outcome Measures | |
|---|---|---|---|---|---|
| Group | Description | Sheep | (In Vivo) | (In Vivo) | (Ex Vivo) |
| 1 | Vertebroplasty | n = 3 | 1 month | Extravasation | Extravasation |
| | treatment with | | | (CT/ | (CT) |
| | adhesive | | | Radiography) | Biomechanical |
| | composition | | | Thrombogenicity | Testing |
| 2 | Vertebroplasty | n = 3 | | (Blood Gas | (Cyclic |
| | treatment with | | | Monitoring) | Loading) |
| | PMMA | | | | |

Surgical Model and Defect Creation: A ventrolateral vertebroplasty procedure was performed on six skeletally mature Rambouillet Columbian ewes (aged 4-6 years). The caudal third of the fourth lumbar vertebral body (L4) was identified by x-ray (lateral plane) for correct longitudinal placement. After a minimally invasive, e.g., percutaneous cut, of 10×10mm was made, a 5mm diameter drill was advanced toward the center of the vertebral body under continuous fluoroscopic guidance observable in two planes. The final depth of the drill channel was defined by the contralateral delimitation of the respective spinous process. The drill was removed and the adhesive composition was delivered by syringe injection. The monitoring and control of such delivery was aided by indirectly using fluoroscopic imaging through the contrast agent present in the adhesive composition. The same procedure was then repeated for the vertebral bodies L2 and L3 to increase sample size. L5 and L6 were used as non-treated controls. A number of sheep were treated with PMMA bone cement for comparison evaluation.

Extravasation Assessment via Computed Tomography and Blood Gas Monitoring: A pre-operative and post-sacrifice full body CT scan were conducted (Siemens Somatom Definition AS 64-slice helical fan-beam CT scanner) to examine the lungs for the formation of pulmonary emboli. Similarly, blood gas and arterial blood pressure were measured (Heska Element POC) at standardized set times immediately before the balloon inflation, 2 minutes after inserting the materials, and during recovery.

Biomechanics Evaluating Stiffness of the Vertebral Body Construct: Following sacrifice, the surrounding soft tissues were removed, and biomechanical evaluations were performed through compressive cyclic loading using a servo-hydraulic material testing machine (858 Mini Bionix, MTS Systems Corporation, Eden Prairie, MN) using the methodology described above.

### Example 6: Percutaneous Treatment of Distal Radius Compression Fracture With Image Guided Delivery of an Adhesive Composition in Human Cadaver

Surgically induced compression fractures were created in human cadaveric distal radii in order to conduct user handling trials with hand and upper extremity surgeons to obtain clinical feedback on how the adhesive composition (TOM250+2.5% BaSO₄+3% PLGA) system would be applied clinically. Distal radius fracture fixations were conducted on bilateral upper extremity specimen pairs from the same cadaver, one using the exemplary adhesive composition, and the other using the current standard of treatment, i.e., standard self-locking volar plates and screws. The percutaneous delivery of an exemplary adhesive composition and the delivery device used to deploy the adhesive composition. Due to the radiopacity of the adhesive compositions, they also assessed the use of fluoroscopy to guide the placement of material in the surgical site and various surgical approaches to applying the therapeutic composition to the wound site as also assessed. The second group of standard treatment using paired specimens allowed comparison of the two groups with the only true difference being the fixation method. This allowed direct comparison of the results.

Biomechanical Evaluation: Fracture Fixation Strength of the Cadaver Specimen: A sub-failure test was completed at a cyclic load of 5 to 50 N to show return to function corresponding to lifting approximately 10 pound weight, with pass/fail criteria being survival of the fixation for 420 cycles following a sinusoidal pattern. Distal radius fractures typically heal in five to six weeks and this sub-failure test regimen demonstrated survival during the healing period with ten load cycles each day. Destructive testing was completed on all test specimens using a servo hydraulic material testing machine (858 Mini Bionix, MTS Systems Corporation, Eden Prairie, MN) to quantify the quasi-static failure load. The proximal radius was fixed at approximately 30 degrees from the vertical, and compression load applied on the proximal margin of the palm approximately against the trapezoid/capitate bone using a spherical actuator. The compressive load was applied under a displacement-controlled method at a fixed rate of 5 mm/s and along the vertical direction until failure of the fracture site. The failure loads of the contralateral paired test specimens were tested for difference using Student's t-test to reject the null hypothesis.

### Example 7: Treatment of an Extremity Osteotomy Wedge Defect in a Sheep Model

Using a published fracture model in sheep, the use of the exemplary adhesive composition alone to internally treat a sheep metaphyseal fracture was compared to metal fixation. The expected primary outcomes of this study were: (a) fixation, confirmed using in vivo gait analysis and ex vivo biomechanical testing, and (b) bone healing examined through cone beam computed tomography and histology evaluation over 3 timepoints (12 weeks, 6 months, 1 year) to assess the initial healing period (12 weeks), full healing across the fracture gap (6 months) and essential bone adhesive material resorption and replacement with new bone (1 year).

Animal Model Justification: The efficacy of the adhesive composition was investigated through short term (12 month) assessment in a clinically relevant sheep model to more closely reflect compression fractures that occur in the extremities. In this model, a full metaphyseal discontinuity wedge-shaped osteotomy was created at the distal femoral metaphysis. The Colorado State University Preclinical Research Laboratory had previously developed an anatomical locking plate which conferred sufficient mechanical stability to the fractured area to allow immediate post-operative full weight bearing. This model has shown metaphyseal bone healing through a direct intramembranous bone formation, which was typical of and unique to the trabecular bone without visible callus and cartilaginous tissue formation. Sheep are considered to have a comparable bone healing rate to humans and have been previously established as useful models for human bone remodeling and turnover activity

Cadaver studies have shown that the human distal radius is subjected to forces of up to 300 N during dart throwing. In comparison, sheep elbow (distal humerus) experiences peak load of 700 N during gait. This force should be comparable, if not greater for the distal femur. Since the cross-sectional area of the human distal radius and the sheep distal femur are comparable, loading of the sheep distal femur from gait produces more than twice the amount of stress than the human distal radius during physiological activities. Therefore, the proposed large animal study represents the worst-case scenario and is a good model for human DRF injuries.

A pilot study was conducted to test the model and obtain an early term assessment of bone healing within the osteotomy site (Table). Subsequently a pivotal study will be conducted to obtain statistically significant results (Table).

TABLE 4
Pilot Sheep Study Design

| | | | Time | Outcome Measures | |
|---|---|---|---|---|---|
| Group | Description | Sheep | (In Vivo) | (In Vivo) | (Ex Vivo) |
| 1 | Adhesive | n = 1 | 8 weeks | Bone bridging | Biomechanical |
| | Composition | | | (radiology) | testing |
| | fixation across | | | | (subthreshold) |
| | osteotomy | | | | |
| | defect | | | | |
| 2 | Metal fixation | n = 1 | | | |
| | without | | | | |
| | Adhesive | | | | |
| | Composition in | | | | |
| | osteotomy site | | | | |

TABLE 5
Pivotal Sheep Study Design

| | | | Time | Outcome Measures | |
|---|---|---|---|---|---|
| Group | Description | Sheep | (In Vivo) | (In Vivo) | (Ex Vivo) |
| 1 | Adhesive | n = 18 | 3 months | Gait analysis | Post-sacrifice |
| | Composition | | (n = 6) | Bone bridging | radiographs |
| | fixation across | | 6 months | (radiology) | Biomechanical |
| | osteotomy | | (n = 6) | | testing (non- |
| | defect | | 12 months | | destructive 3- |
| 2 | Metal fixation | n = 18 | (n = 6) | | point bending |
| | without | | | | Micro-CT |
| | Adhesive | | | | Histology |
| | Composition in | | | | |
| | osteotomy site | | | | |

Surgical Model and Defect Creation: Skeletally mature Rambouillet Columbian ewes (age 4-6 years) were used in this study. An ostectomy procedure was performed by creating a wedge-shaped ostectomy in the right distal femur metaphysis. An exemplary adhesive composition was injected percutaneously through a minimally invasive, image-guided, surgical procedure to fixate the fractured bone. The efficacy of the adhesion of the adhesive composition and the efficacy of the image-guided percutaneous delivery of the adhesive composition were assessed. The adhesive composition included PLGA microfibers for additional strength, nanoparticles of calcium carbonate were for additional porosity, and barium sulfate was added as an imaging agent. The adhesive composition was delivered percutaneously via a cannula tip while monitoring the delivery via fluoroscopic imaging of the barium sulfate in the adhesive composition. All wounds were closed in layers and covered with spray dressing, sterile compresses, and elastic conforming bandages. Finally, radiographs were made immediately postoperatively.

Analysis and Postmortem Procedure: All sheep were assessed in vivo for gait function which will be assessed pre-operatively, and every six (6) weeks until necropsy. Radiographs to assess bone bridging were made on a routine basis. Sheep were euthanized at the study timepoints, and each limb was harvested. The repaired ostectomy site and contralateral femur of each sheep were explanted with preservation of the surrounding soft tissues. Explanted specimens were tested for biomechanical stiffness (sub-threshold), preserved in formalin, and later subjected to histological examination to assess bone fusion.

Micro-Computed Tomography: Post-sacrifice micro-computed tomography (μCT) analyses were performed at the fracture location of each group (Scanco μCT 80, Sanco Medical AG, Bruttisellen, Switzerland). A uniform region of interest (ROI) centered within the fracture defect and extending to the cranial and caudal surfaces of the native bone was created. Bone volume (BV, mm³), bone volume fraction (BV/TV, %) and BMD (mg HA/cm³) were analyzed for each specimen and compared to contralateral controls.

Biomechanics Evaluating Stiffness of the Bone-Implant Construct: Following sacrifice, the surrounding soft tissues were removed, and biomechanical evaluations were performed on the treated distal femur of each group. Non-destructive three-point bending experiments were performed on the dissected bones using a servo-hydraulic material testing machine (858 Mini Bionix, MTS Systems Corporation, Eden Prairie, MN). The actuator will be lowered at a rate of 1.0 mm/sec to 200 N or until the specimen [symbol] maximum elastic deflection was achieved (i.e., prior to inducing permanent deformation). The sample was preconditioned five times and the load displacement data from the final cycle was utilized to calculate the specimen bending stiffness.

Histomorphometric Analysis: After biomechanical testing, the fracture region was explanted. Each bone specimen was processed undecalcified histological analysis. Samples were stored in 70% ethanol until fully fixed. Samples were dehydrated in graded solutions of ethanol and cleared with acetone and infiltrated with a series of three solutions containing Acrylosin and a catalyst of PERKADOX®16 (Dorn and Hart Microedge, Villa Park, IL) over the course of approximately 20 days. Upon completion, the specimens were polymerized into a hardened block.

Histological sections were taken in the transverse, i.e., mediolateral, plane to include the fracture site and any associated callus of the distal femur. Two sections were cut from each specimen using an Exakt diamond blade bone saw (Exakt Technologies, Oklahoma City, OK) at a thickness of 300-400 μm, and ground using an Exakt micro grinder to a thickness of approximately 50 μm. One section from each specimen was stained with Sanderson [symbol] Rapid Bone Stain and counter-stained with Van Gieson's stain, while the other section will remain unstained for dynamic histomorphometric analysis. High-resolution digital images were acquired for histomorphometric analysis using a Spot Imaging system (Diagnostic Instruments, Sterling Heights, MI), a Nikon E800 microscope (AG Heinze, Lake Forest, CA), and analyzed with an Image Pro imaging system (Media Cybernetics, Silver Spring, MD). For static histomorphometric analysis, stained sections were analyzed for percent bone, soft tissue, implant, and void areas within the healing fracture. For dynamic histomorphometry, three fluorescent images were acquired per specimen (at 40× magnification) within the healing fracture and mineralizing surface (MS), mineral apposition rate (MAR), and bone formation rate (BFR) were be calculated.

1. A method of image-guided delivery of an adhesive composition to a wound site, the method comprising:
i) acquiring image data of the wound site;
ii) positioning a device to deliver the adhesive composition to the bone site; and
iii) delivering the adhesive composition to the bone site via the device.

2. A method of joining two or more bone fragments together at a wound site, the method comprising:
i) acquiring image data of the wound site;
ii) positioning a device constructed and arranged to deliver an adhesive composition to the wound site; and
iii) delivering the adhesive composition to the wound site via the device.

3. The method of claim 1 or 2, wherein the positioning of the delivery device is responsive to the image data acquired.

4. The method of any one of claim 1 or 2, wherein the delivering of the adhesive composition is responsive to the image data acquired.

5. The method of any one of claim 1 or 2, wherein delivering the adhesive composition further comprises controlling the delivery of the adhesive composition to the wound site.

6. The method of claim 5, wherein controlling the delivery of an adhesive composition comprises controlling one or more of: the amount of adhesive composition delivered, the location of delivery of the adhesive composition, the duration of delivery of the adhesive composition, or the flow rate of delivery of the adhesive composition.

7. The method of any one of claim 1 or 2, wherein delivering the adhesive composition further comprises delivering an amount of the adhesive composition sufficient to prevent leakage of the adhesive composition from the wound site.

8. The method of any one of claim 1 or 2, further comprising comparing the amount of the adhesive composition delivered to the wound site to a desired quantity of the adhesive composition to be delivered to the wound site.

9. The method of any one of claim 1 or 2, wherein the acquiring of i) comprises directly acquiring the image data or indirectly acquiring the image data

10. The method of any one of claim 1 or 2, wherein i) is performed prior to ii).

11. The method of any one of claim 1 or 2, wherein i) is performed prior to iii).

12. The method of any one of claim 1 or 2, wherein i) is performed after ii) but before iii).

13. The method of any one of claim 1 or 2, wherein i) is performed after ii) and iii).

14. The method of any one of claim 1 or 2, wherein the acquiring of i) comprises:
acquiring a first set of image data of the wound site prior to delivering the adhesive composition to the wound site; and
acquiring a second set of image data of the wound site after delivering the adhesive composition to the wound site.

15. The method of claim 9, further comprising acquiring a third set of image data of the wound site while delivering the adhesive composition to the wound site.

16. The method of any one of claim 1 or 2, wherein acquiring image data comprises directly acquiring the image data or indirectly acquiring the image data.

17. The method of any one of claim 1 or 2, wherein acquiring image data comprises acquiring image data via a two-dimensional imaging method or a three-dimensional imaging method.

18. The method of any one of claim 1 or 2, wherein acquiring of image data comprises acquiring data related to heat, position of sound waves, position of X-rays, position of radio waves, and/or the presence of a contrast agent.

19. The method of any one of claim 1 or 2, wherein acquiring of image data comprises at least one of fluoroscopy, mechanical tomography, computed tomography (CT), magnetic resonance imaging, X-ray, ultrasound, positron emission tomography (PET), or scintigraphy.

20. The method of claim 18, wherein the acquiring image data comprises fluoroscopy.

21. The method of any one of claim 1 or 2, wherein acquiring image data comprises operating an ultrasound wand, a fluoroscope, mechanical tomography equipment, or an X-ray generator.

22. The method of any one of claim 1 or 2, wherein acquiring of image data comprises use of a computer-assisted imaging system.

23. The method of claim 22, wherein use of a computer-assisted imaging system comprises:
a) acquiring signal data from a wound site;
b) converting the signal data to image data; and/or
c) displaying the image data to a user.

24. The method of any one of claim 1 or 2, wherein the adhesive composition comprises a multivalent metal salt, an organic compound, and an aqueous medium.

25. The method of claim 24, wherein the multivalent metal salt comprises tetracalcium phosphate or tricalcium phosphate.

26. The method of claim 24, wherein the organic compound is a compound of any one of Formulas (I), (II), (III), (IV), (V), or (VI).

27. The method of claim 26, wherein the organic compound is a compound of Formula (I): 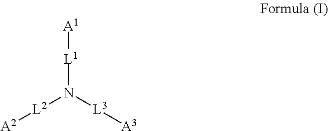
or a salt thereof, wherein:
each of A¹, A², and A³ is independently selected from an acidic group; and
each of L¹, L², and L³ is independently a bond, alkylene, or heteroalkylene.

28. The method of claim 26, wherein the organic compound is a compound of Formula (II): 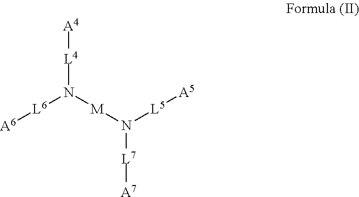
or a salt thereof, wherein:
each of A⁴, A⁵, and A⁶ is independently selected from an acidic group;
A⁷ is selected from an acidic group, a hydrogen atom, an alkyl, an aryl, a hydroxy group, a thio group, and an amino group;
each of L⁴, L⁵, L⁶, and L⁷ is independently a bond, alkylene, or heteroalkylene; and
M is alkylene or heteroalkylene.

29. The method of claim 26, wherein the organic compound is a compound of Formula (III): 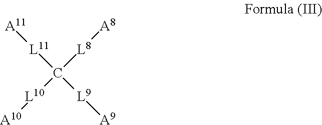
or a salt thereof, wherein:
each of A⁸ and A⁹ is independently selected from an acidic group;
each of A¹⁰ and A¹¹ is independently selected from an acidic group, a hydrogen atom, an alkyl, aryl, a hydroxy group, a thio group, and an amino group;
each of L⁸, L⁹, L¹⁰ and L¹¹ is independently a bond, alkylene, or heteroalkylene.

30. The method of claim 26, wherein the organic compound is a compound of Formula (IV): 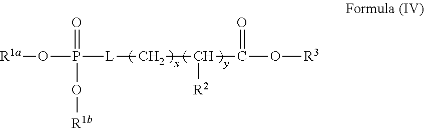
or a salt thereof, wherein:
L is O, S, NH, or CH2;
each of R^{1a} and R^{1b} is independently H, an optionally substituted alkyl, or an optionally substituted aryl;
R² is H, NR^{4a}R^{4b}, C(O)R⁵, or C(O)OR⁵;
R³ is H, an optionally substituted alkyl, or an optionally substituted aryl;
each of R^{4a} and R^{4b} is independently H, C(O)R⁶, or an optionally substituted alkyl;
R⁵ is H, an optionally substituted alkyl, or an optionally substituted aryl;
R⁶ is an optionally substituted alkyl or an optionally substituted aryl; and
each of x and y is independently 0, 1, 2, or 3.

31. The method of claim 26, wherein the organic compound is a compound of Formula (V): 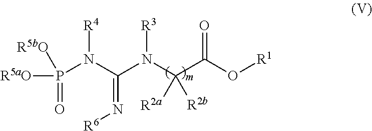
or a salt thereof, wherein:
R¹ is H, optionally substituted alkyl, optionally substituted alkenyl, optionally substituted alkynyl, optionally substituted aryl, or optionally substituted heteroaryl;
each of R^{2a} and R^{2b} is independently H, optionally substituted alkyl, hydroxy, alkoxy, or halo;
each of R³ and R⁴ is independently H or optionally substituted alkyl;
each of R^{5a} and R^{5b} is independently H, optionally substituted alkyl, optionally substituted alkenyl, optionally substituted alkynyl, optionally substituted aryl, or optionally substituted heteroaryl;
R⁶ is H or optionally substituted alkyl; and
m is 1, 2, 3, 4, or 5.

32. The method of claim 26, wherein the compound is a compound of Formula (VI): 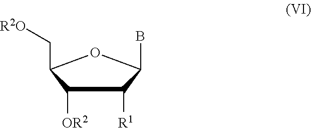
or a salt thereof, wherein:
B is a nucleobase;
R¹ is H, OR⁴, or halo;
R² is H, optionally substituted alkyl, optionally substituted alkenyl, optionally substituted alkynyl, optionally substituted aryl, optionally substituted heteroaryl, optionally substituted cycloalkyl, or optionally substituted heterocyclyl;
p1 R³ is H, optionally substituted alkyl, or a phosphate moiety (e.g., monophosphate or diphosphate); and
R⁴ is H, optionally substituted alkyl, optionally substituted alkenyl, optionally substituted alkynyl, optionally substituted aryl, optionally substituted heteroaryl, optionally substituted cycloalkyl, or optionally substituted heterocyclyl.

33. The method of any one of claim 1 or 2, wherein the adhesive composition has a viscosity between 100 cP and 10,000 cP.

34. The method of any one of claim 1 or 2, wherein the adhesive composition further comprises an agent (e.g., a contrast agent or radioisotope).

35. The method of claim 34, wherein the agent is a contrast agent.

36. The method of claim 34, wherein the amount of the agent in the adhesive composition is less than about 20%, 15%, 10%, 9%, 8%, 7%, 6%, 5%, 4%, 3%, 2%, 1%, 0.5%, 0.1%, 0.05%, or 0.01% (w/w or w/v) of the total adhesive composition.

37. The method claim of 35, wherein the contrast agent comprises barium, iodine, bismuth, gadolinium, CO₂, hydrogen, or air.

38. The method of claim 35, wherein the contrast agent is formulated as a liposome.

39. The method of claim 35, wherein the contrast agent is BaSO₄.

40. The method of any one of claim 1 or 2, wherein the adhesive composition has an adhesive strength upon curing of about 250 kPa or more.

41. The method of any one of claim 1 or 2, further comprising iv) making an incision in a subject.

42. The method of claim 41, wherein the making of the incision is responsive to acquiring image data.

43. The method of claim 41, wherein the delivering of the adhesive composition occurs following making the incision in the subject.

44. The method of any one of claim 1 or 2, wherein the delivering of the adhesive composition comprises delivering the adhesive composition percutaneously via an incision.

45. The method of any one of claim 1 or 2, wherein delivering of the adhesive composition comprises delivering the adhesive composition to the wound site via a lumen of the device, the lumen being defined within a wall of the device having an outer dimension between about 5 mm to about 10 mm.

46. The method of claim 45, wherein a length of the wall of the lumen is about 30 mm to about 300 mm.

47. The method of any one of claim 1 or 2, further comprising referencing one or more fiducial features on one or more components of the device.

48. The method of any one of claim 1 or 2, wherein the delivery system further comprises one or more cannula tips for delivering the adhesive composition to the wound site.

49. The method of claim 48, further comprising providing an alert when the device has reached the wound site.

50. The method of any one of claims 1-48, further comprising controlling one or more of the timing, direction, volume, or rate of the delivery of the adhesive composition.

51. The method of any one of claim 1 or 2, wherein the device is constructed and arranged to direct a flow of the adhesive composition at an angle relative to a long axis of the device.

52. The method of any one of claim 1 or 2, wherein the device is constructed and arranged to direct a flow of the adhesive composition concentrically and parallel to its axis.

53. The method of any one of claim 1 or 2, wherein the cannula tip comprises a detectable registration feature used to indicate the trajectory opening or path of the adhesive composition during delivery.

54. The method of any one of claim 1 or 2, wherein the delivery system further comprises a balloon or mesh containment device.

55. The method of claim 54, wherein the balloon or mesh containment device is microporous.

56. The method of any one of claim 1 or 2, wherein the adhesive composition is a therapeutic composition.

57. The method of any one of claim 1 or 2, wherein the adhesive composition stimulates bone regeneration.

58. The method of any one of claim 1 or 2, wherein the adhesive composition is a pain control agent.

59. The method of any one of claim 1 or 2, wherein the adhesive composition is antimicrobial.

60. An image guided delivery system comprising a delivery device and an adhesive composition, e.g., as described herein.
